(19) United States
(12) Patent Application Publication
(10) Pub. No.: US 2026/0306691 A1
(43) Pub. Date: Oct. 8, 2026

(54) DISPENSER FOR A VOLATILE LIQUID COMPOSITION

(71) Applicant: THE PROCTER & GAMBLE COMPANY, Cincinnati, OH (US)

(72) Inventors: Ronald David TURNER, Walton, KY (US); Hua HU, Mason, OH (US); Bryn Harper GERLACH, Newtown, OH (US)

(21) Appl. No.: 19/638,153

(22) Filed: Apr. 3, 2026

Related U.S. Application Data
(60) Provisional application No. 63/783,270, filed on Apr. 4, 2025.

(51) Int. Cl. A61L 9/03

(52) U.S. Cl. CPC A61L 9/037; A61L 2209/133; A61L 2209/134

(57) ABSTRACT

A dispenser for a volatile liquid composition includes a housing comprising an air inlet opening and an air outlet opening, the housing being configured to contain a reservoir and a delivery engine, and an evaporative assistance element, where the evaporative assistance element defines a channel. The dispenser defines an upward air flow path from the inlet opening through the channel and through the outlet opening. The dispenser has improved fragrance distribution.

### FIELD OF THE INVENTION

The present disclosure is in the field of dispensers for volatile liquid compositions and related methods.

### BACKGROUND OF THE INVENTION

Volatile composition dispensers exist for delivering various volatile compositions, such as freshening compositions, into the air. Such volatile composition dispensers may, for example, take the form of a wick-based electrical dispenser having one or more heaters to assist with volatilizing the volatile composition into the air.

Plug-in air fresheners, such as those marketed under the brand name Febreze®, have become popular solutions for enhancing indoor air quality by emitting pleasant fragrances and neutralizing unwanted odors. These devices typically utilize a liquid fragrance formulation that is evaporated into the air via a heating element or passive diffusion. However, several significant challenges remain in achieving an optimal aromatic environment within a room.

Current plug-in air fresheners can struggle to achieve uniform fragrance distribution throughout a room. Due to their localized placement and the nature of diffusion, the scent may be concentrated in the immediate vicinity of the device while leaving other areas of the room inadequately perfumed. This can create an uneven aromatic experience, leading to user dissatisfaction. Many plug-in devices are designed for use in standard-sized rooms, which can limit their effectiveness in larger or irregularly shaped spaces. The fragrance may not reach all corners or elevated areas within the room, resulting in a patchy scent experience. The rate of evaporation of fragrance from plug-in devices can vary based on environmental conditions such as temperature and humidity. This variability can lead to inconsistent fragrance intensity over time, with the scent becoming too strong initially and fading quickly afterward.

Thus, there is a need for an improved method and apparatus for the dispensing of a volatile liquid composition, preferably a fragrance in a room using an electric air freshener, that addresses the aforementioned challenges.

### SUMMARY OF THE INVENTION

In an example, a dispenser for dispensing a volatile liquid composition is provided. The dispenser comprises a housing comprising an air inlet opening and an air outlet opening and an evaporative assistance element that defines a channel. The dispenser defines an upward air flow path from the inlet opening through the channel and through the outlet opening, i.e., the inlet opening is placed below the channel and the channel is placed below the outlet opening. The dispenser can also include a reservoir for containing the liquid composition and a delivery engine in fluid communication with the volatile liquid composition, wherein the delivery engine comprises an evaporative surface at least partially extending through the channel. This configuration allows for an air flow path that promotes air intake from the air inlet opening that then goes through the channel and then leaves the housing through the air outlet opening, promoting the delivery of the volatile liquid composition. The channel may have a cross-section area (A_{channel}) in a range of, for example, about 35 mm² to about 65 mm². The air inlet opening may have an area (Aᵢₙₗₑₜ) in a range of, for example, about 400 mm² to about 600 mm². A ratio of a length of the evaporative surface overlapped by the evaporative assistance element to a shortest distance between the air inlet opening to the channel entrance may be greater than 0.6, preferably greater than 0.7. A ratio of a flow resistance through the air inlet flow path to a flow resistance through the channel may be less than 0.05, preferably less than 0.02.

The evaporative assistance element may surround at least 70%, preferably at least 80%, more preferably at least 90%, on a length basis, of the evaporative surface.

In another example, a dispenser for dispensing a volatile liquid composition is provided. The dispenser comprises a housing, an air inlet opening, and an air outlet opening. The housing retains: a) a reservoir holding the liquid composition; and b) a delivery engine in fluid communication with the liquid composition, the delivery engine comprises an evaporative surface; and c) an evaporative assistance element. The evaporative assistance element surrounds the evaporative surface to define a channel. The inlet opening, the channel and the outlet opening are part of an upwards air flow path, i.e., the inlet opening is placed below the channel and the channel is placed below the outlet opening. This configuration allows for an air flow path that promotes air intake from the air inlet opening that then goes through the channel and then leaves the housing through the air outlet opening, promoting the delivery of the volatile liquid composition.

The ratio of the length of the evaporative surface surrounded by the evaporative assistance element to the shortest distance between the air inlet opening to the channel entrance may be greater than 0.6 preferably greater than 0.7.

A method for delivering a volatile liquid composition, preferably a fragrance, using a dispenser as disclosed herein is also provided.

### BRIEF DESCRIPTION OF DRAWINGS

The above-mentioned and other features and advantages of the present disclosure, and the manner of attaining them, will become more apparent and the present disclosure itself will be better understood by reference to the following description of various examples of the present disclosure taken in conjunction with the accompanying drawings, wherein:

FIG. 1 is a perspective view of an example volatile composition dispenser.

FIG. 2 is a view of the front and top of the volatile composition dispenser shown in FIG. 1.

FIG. 3 is a view of the side and back of the volatile composition dispenser shown in FIG. 1.

FIG. 4 is a schematic, exploded view of the volatile composition dispenser of FIG. 1.

FIG. 5 is a cross-sectional view of the volatile composition dispenser shown in FIG. 1.

FIG. 6 is a cross-sectional view of the volatile composition dispenser of FIG. 1 showing an air flow path.

FIG. 7 is a cross-sectional view of the volatile composition dispenser of FIG. 1 showing various dimensions.

FIG. 8 is a top perspective view of an example volatile composition dispenser.

FIG. 9 is a bottom perspective view of the volatile composition dispenser shown in FIG. 8 including a volatile composition cartridge.

FIGS. 10A-10I are Background Oriented Schlieren (BOS) photographs showing plumes during use of various volatile composition dispensers.

FIGS. 11A-11C are charts showing the weight loss of a volatile composition on different heat settings over time.

### DETAILED DESCRIPTION OF THE INVENTION

Various examples will now be described to provide an overall understanding of the principles of the structure, function, manufacture, and use of the dispensers and methods disclosed herein. One or more of these examples are illustrated in the accompanying drawings. Those of ordinary skill in the art will understand that the devices and methods specifically described herein and illustrated in the accompanying drawings are non-limiting examples and that the scope of the various examples of the present disclosure is defined solely by the claims. The features illustrated or described in connection with one example can be combined with the features of other examples. Such modifications and variations are intended to be included within the scope of the present disclosure.

A volatile composition dispenser can be used to dispense a volatile liquid composition, such as a fragrance, an insecticide, or any other volatile liquid composition, including those that help with sleep, breathing, coughing, congestion, etc., to an area surrounding the dispenser. Preferably, the volatile liquid composition is a fragrance. The dispersion of the volatile composition can be significantly enhanced in terms of consistency and total dispersion via the addition of strategically arranged air inlet openings (herein also referred as “vents” and/or “apertures”) to the volatile composition dispenser as discussed in more detail below. Through addition of openings on the sides, front, and/or back of the housing to improve airflow within the dispenser, one can impact the room fill (both speed and consistency of experience). Surprisingly, this increase in dispersion does not have a significant impact on the overall evaporation rate. Thus, the dispensing effect of the volatile composition dispenser is improved without needing to more frequently replace or refill the volatile composition reservoir.

### Dispenser

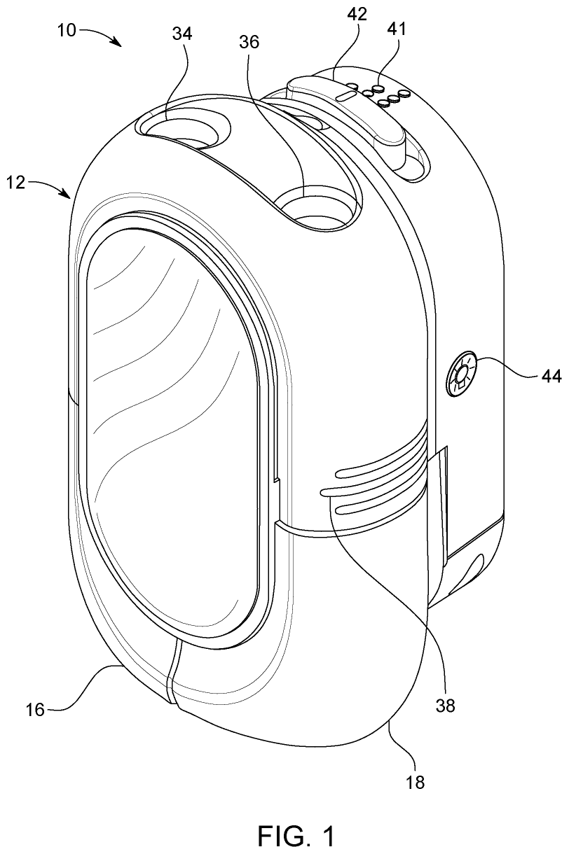

With reference to FIGS. 1-7, an example volatile composition dispenser 10 may take the form of an electrical wall plug volatile composition dispenser. The volatile composition dispenser 10 may include a housing 12 and an electrical plug 14. The electrical plug 14 may be directly or indirectly coupled to the housing 12 or may be integral with the housing 12. The volatile composition dispenser 10 may be configured to plug directly into a wall outlet such that the housing 12 is supported by the electrical plug 14. In another example, the volatile composition dispenser 10 may be configured with a power cord, replaceable battery, or rechargeable battery such that the volatile composition dispenser 10 rests on a horizontal surface, such as a table, countertop, desktop, appliance, or the like. A power cord may have a length of, for example, at least about 10 cm, at least about 20 cm, at least about 30 cm, at least about 50 cm, from about 10 to about 300 cm, from about 90 to about 300 cm, from about 90 to about 200 cm, from about 150 to about 200 cm, or any values within the foregoing ranges or any ranges created thereby. Suitable volatile composition dispensers are described in U.S. Pat. No. 11,896,743 and U.S. Patent App. Pub. No. 2025/0319224, which are each incorporated herein by reference in their entireties.

The volatile composition dispenser 10 may include at least one reservoir for containing the volatile composition(s). For illustrative purposes, a first reservoir 16 and a second reservoir 18 are shown in FIG. 1. The housing 12 may serve as a holder for the reservoirs 16, 18 and any of the other components of the volatile composition dispenser 10. The reservoirs 16, 18 may contain a first volatile composition 20 and a second volatile composition 22, respectively.

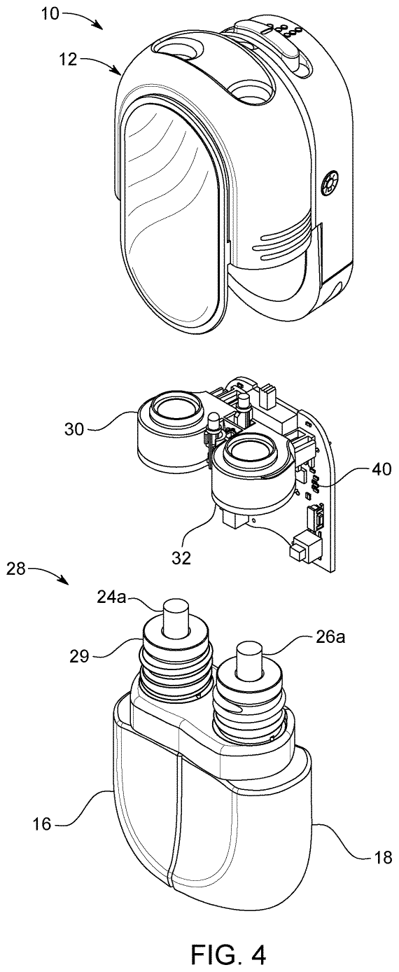

The volatile composition dispenser comprises one or more delivery engines that, in use, transports the volatile composition out of the reservoir. The delivery engine has an evaporative surface in fluidic communication with the volatile composition in the reservoir. For illustrative purposes, a first delivery engine 24 and a second delivery engine 26 are shown as wicks in FIGS. 4-7. A first portion of each wick extends into the respective reservoir, and a second, remaining portion of the wick extends out of the respective reservoir, such as through the top of the reservoir. The exterior surface of each wick (e.g., 24, 26) that is exposed to the environment outside of the reservoir 16, 18 is an evaporative surface (e.g., 24a, 26a). The delivery engine(s) and reservoir(s) may be in the form of a cartridge. For example, volatile composition dispenser 10 includes a cartridge 28 including the reservoirs 16, 18 and delivery engines 24, 26. In use, the volatile composition travels from the reservoir through the delivery engine and evaporates from the evaporative surface into an interior of the housing. Air flow through the housing carries the evaporated composition out of the volatile composition dispenser.

The reservoirs may comprise a seal 29, such as shown in FIG. 4, for containing the volatile composition. The volatile composition dispenser and/or the reservoir(s) may further comprise an additional seal for covering the wick when the volatile composition is not being emitted.

The volatile composition dispenser includes one or more evaporative assistance elements for assisting with the volatilization of the volatile compositions from the evaporative surfaces. The evaporative assistance element may be placed adjacent to the evaporative surface. For illustrative purposes, heaters 30, 32 are shown in FIGS. 4-7 that comprise heating elements in the form of circular or annular rings that at least partially surround the wicks protruding from the bottles of the volatile compositions. The heaters 30, 32 are adjacent at least a portion of the evaporative surfaces 24a, 26a of the delivery engines 24, 26, respectively. The evaporative assistance elements 30, 32 define channels 30a, 32a. When the cartridge 28 is removably coupled to the housing 12, an upper portion of the wick 24, 26 extends into the channel 30a, 32a of the evaporative assistance element 30, 32.

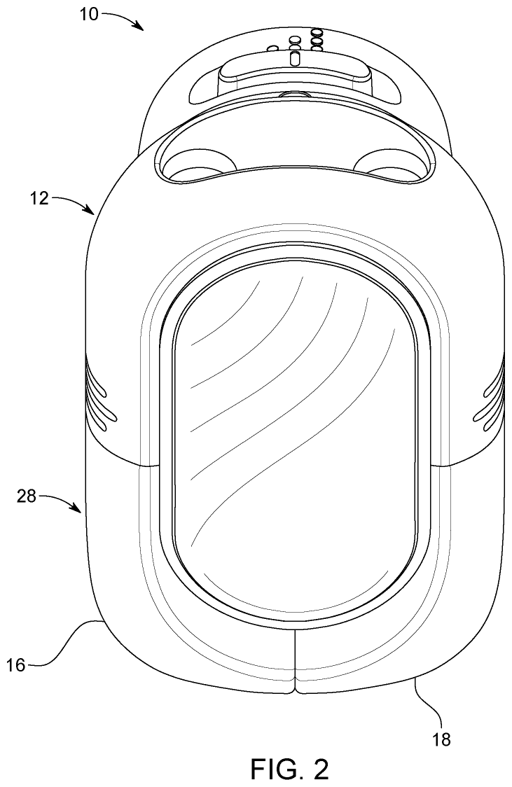

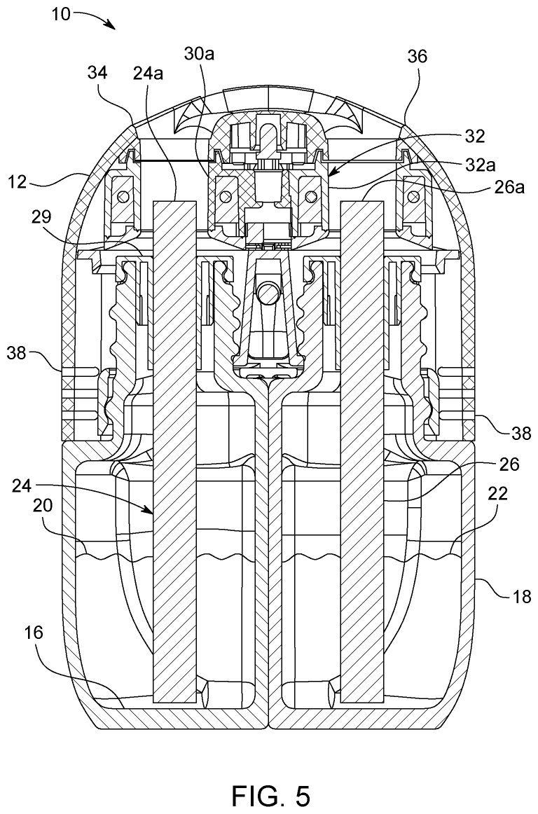

With further reference to FIGS. 1-4, the housing 12 includes a top, bottom, front, rear, a first side, and a second side opposite the first side. The housing may include at least one air outlet, which is in fluid communication with the evaporative surface. As best seen in FIGS. 2 and 5, the dispenser 10 includes a first air outlet 34 and a second air outlet 36. The air outlets 34, 36 are in fluid communication with the evaporative surfaces 24a, 26a, respectively. Additionally, apertures or vents 38 are defined in the housing 12. As illustrated in FIG. 1, the vents 38 comprise three discrete apertures arranged horizontally on the first and second sides of the housing 12. As described below, the orientation, size, arrangement, and location of the vents 38 may vary. As further described below, air moves into the dispenser 10 through the vents 38, passes over the evaporative surfaces 24a, 26a, and exits the dispenser 10 through the air outlets 34, 36.

The dispenser can comprise a sensor, such as a visible indicator, a light source, and/or an audible alert, configured to provide feedback to the user regarding the status of the dispenser. For example, as illustrated in FIG. 1, a series of lights 41 may indicate a setting of the dispenser 10 (e.g., one light for Low, two lights for Medium, and three lights for High).

The volatile composition dispenser may include one or more buttons or selectors (e.g., a dial) for adjusting the energy supplied to the evaporative assistance element(s) or another function of the volatile composition dispenser. As illustrated in FIG. 1, a sliding selector 42 may be moved by a user to adjust a heat setting of the volatile composition dispenser 10. Also illustrated is a button 44 for controlling a light source (not shown) of the volatile composition dispenser 10. As another example, the volatile composition dispenser may include an energy boost button. An energy boost at various extended emission periods over the life of the volatile composition in the reservoir can increase noticeability of the volatile composition over the use of the dispenser by maintaining or increasing the evaporation rate of the volatile composition from the evaporative surface over time. The buttons or selectors may be on the device and/or be a part of an application used to remotely control the device. The application may be accessed via a mobile device, such as a phone or other computing device.

While FIGS. 1-7 illustrate two reservoirs, two evaporative assistance elements, and two delivery engines, it is to be appreciated that a volatile composition dispenser may include one, two, three, or more reservoirs. Each reservoir in a volatile composition dispenser may include a separate delivery engine. A single evaporative assistance element may be used for one or more evaporative surfaces or each evaporative surface may be adjacent to a unique evaporative assistance element. A single evaporative surface may be adjacent more than one evaporative assistance element. If the volatile composition dispenser includes more than one reservoir, each reservoir may contain a different volatile composition or may contain the same volatile composition. The distance between evaporative surfaces may vary. For example, a distance between wicks may be in a range of about 5 mm to about 40 mm or about 12 mm to about 20 mm.

If one reservoir is present, the volatile composition dispenser may include one, two, or more than two delivery engines that are each in fluid communication with the one reservoir and one, two, or more evaporative surfaces that are in fluid communication with the delivery engines. In such a configuration, the volatile composition dispenser may include one or more evaporative assistance elements. If more than one delivery engine is in fluid communication with a single reservoir, then each delivery engine may be used to volatilize the same volatile composition. This configuration may allow for each delivery engine, such as a wick, to have an extended period where the evaporative assistance element is either delivering low energy or is OFF, giving each delivery engine time for the volatile composition to drain and potentially unclog from the delivery engine. Such a configuration may be particularly useful where the delivery engines are in the form of wicks, which can suffer from wick-clogging of components of volatile compositions.

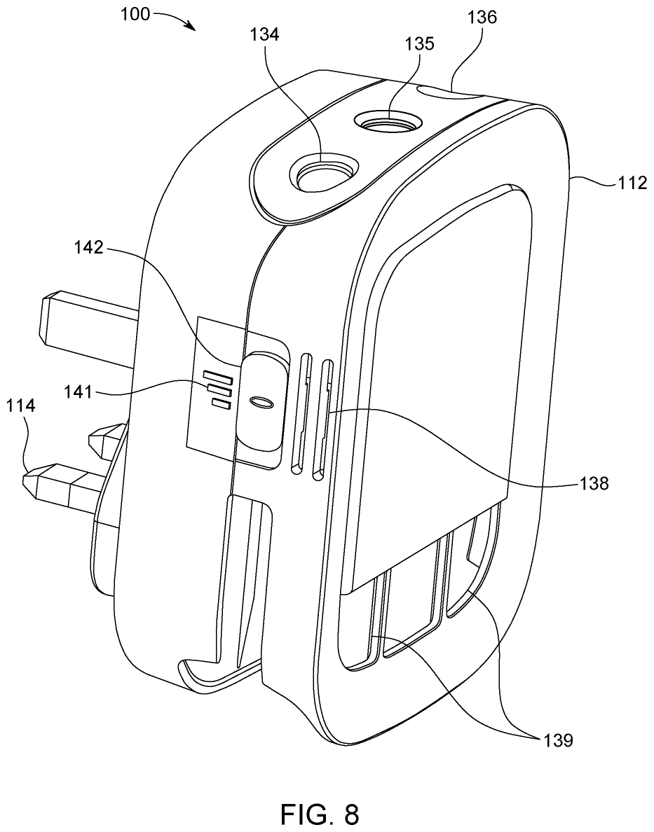

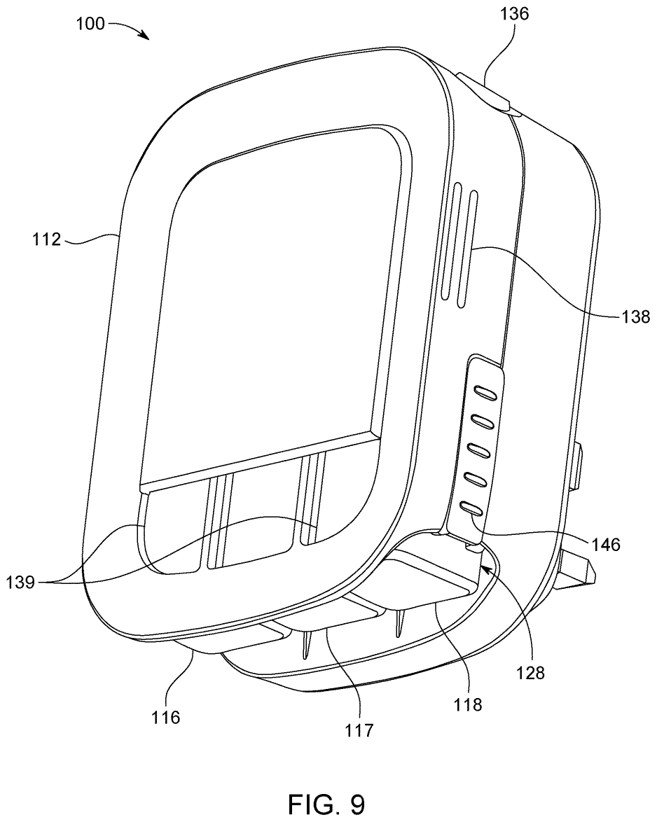

With reference to FIGS. 8 and 9, an example volatile composition dispenser 100 is shown with a first reservoir 116, a second reservoir 117, and a third reservoir 118. The volatile composition dispenser 100 may include a housing 112, an electrical plug 114, a sliding selector 142 for adjusting a heat setting of the volatile composition dispenser 100, and a series of lights 141 for indicating the heat setting in use. The housing 112 may serve as a holder for the reservoirs 116, 117, 118 and any of the other components of the volatile composition dispenser 100. The reservoirs 116, 117, 118 are connected by a collar 146 to be in the form of a cartridge 128. The collar 146 may be used to grip the cartridge 128 for insertion and removal of the cartridge 128. In use, the volatile composition travels from the reservoirs 116, 117, 118 through the delivery engine (not shown) and evaporates from the evaporative surfaces (not shown) into an interior of the housing 112. As illustrated in FIG. 8, the housing 112 includes vents 138 in addition to windows 139. The vents 138 comprise two discrete apertures arranged vertically on the first and second sides of the housing 112. Air flow through the housing 112 carries the evaporated composition out of the first, second, and third air outlets 134, 135, 136 of the volatile composition dispenser 100.

### Housing

As previously described, the dispenser comprises a housing. The housing may be configured to contain many or all of the elements of the dispenser (e.g., reservoir(s), delivery engine(s), and evaporative assistance element(s)). The housing helps to protect internal components from physical damage and exposure to dust and moisture and to prevent unintentional user contact with the electrical and heating elements inside the housing. The housing may be comprised of a single element or from multiple elements that are removably or permanently coupled. The housing defines an interior, which may have one or multiple interior spaces or chambers. The dispenser 10 shown in FIG. 1 has an interior including two adjacent chambers. The housing may take any shape and may be composed of various materials, such as plastic, metal, resin, and the like. The housing may be made of recyclable or sustainable materials.

The dispenser may be configured for the reservoir, delivery engine, and/or cartridge to be removably coupled with the housing. A user can then replenish the reservoir and delivery engine once the volatile composition has been evaporated. The housing can optionally comprise a movable door (not shown) that can be opened to remove and/or replace the reservoir or cartridge.

### Reservoir

The reservoir may be configured to contain from about 5 milliliters (mL) to about 100 mL, alternatively from about 10 mL to about 50 mL, alternatively from about 15 mL to about 30 mL of fluid composition. The dispenser may be configured to have multiple reservoirs, with each reservoir containing the same or different fluid composition and same or different fluid volumes.

The reservoir can be made of any suitable material for containing a fluid composition including glass, plastic, metal, or the like. The reservoir may be made of recyclable or sustainable materials. The reservoir may be transparent, translucent, or opaque or any combination thereof. For example, the reservoir may be opaque with a transparent indicator of the level of fluid composition in the reservoir.

### Delivery Engine

The delivery engine is in fluid communication with the volatile liquid composition. In examples, part of the delivery engine is in the reservoir and part of the delivery engine is outside of the reservoir during use. The part of the delivery engine outside of the reservoir is herein referred to as an evaporative surface. The evaporative surface may be partially or entirely surrounded by the evaporative assistance element. The evaporative assistance element may surround at least 70%, preferably at least 80%, more preferably at least 90%, and up to 100%, on a length basis, of the evaporative surface. The evaporative surface may be located directly above the reservoir.

Instead of a wick, the delivery engine may include a breathable membrane. A volatile composition dispenser may comprise a cartridge having a liquid reservoir for containing a volatile composition and a delivery engine in the form of a breathable membrane enclosing the liquid reservoir, such as disclosed in U.S. Pat. Nos. 8,709,337 and 8,931,711, which are incorporated herein by reference in their entireties. The volatile composition dispenser may also include an evaporative assistance element in the form of a fan. As used herein, a breathable membrane is a vapor permeable membrane that prevents free flow of liquid out of the membrane, thus addressing leakage problems.

Suitable breathable membranes include, but are not limited to, UHMWPE-type membrane optionally filled with silica as described in U.S. Pat. No. 7,498,369, which is hereby incorporated herein in its entirety. The delivery engine may also include a rupturable substrate that seals the volatile composition in the liquid reservoir until a rupture mechanism is engaged to when the volatile composition dispenser is to be used by the consumer, such as disclosed in U.S. Pat. No. 8,709,337. When the consumer is ready to use the volatile composition dispenser, the consumer can rupture the rupturable substrate with the rupture mechanism, which allows the volatile composition in the liquid reservoir to contact the breathable membrane.

The evaporative surface may be configured in various ways. For example, the evaporative surface may be in the form of a wick, membrane, gel, wax, porous or semi-porous substrate, including a felt pad. A dispenser can have one or more reservoir and one or more evaporative surfaces. Evaporative surfaces of different geometries are suitable for use herein, including spiral, rectangular, cylindrical, etc. Preferred evaporative surfaces for use herein have a cylindrical shape. Especially preferred for use herein are cylindrical wicks.

The wick may be defined by a length and a diameter or width, depending on the shape. The wick may have various lengths. For example, the length of the wick may be in the range of about 1 millimeter (“mm”) to about 100 mm, or from about 5 mm to about 75 mm, or from about 10 mm to about 50 mm. The wick may have various diameters or widths. For example, diameter or width of the wick may be at least 1 mm, or at least 2 mm, or at least 3 mm, or at least 4 mm or may be in a range of about 1 mm to about 20 mm, or about 5 mm to about 10 mm. A wick may exhibit a density. The wick density may be in the range of about 0.100 grams/cm³ (“g/cc”) to about 1.0 g/cc.

The term “percent on a length basis” as used herein means that the percentage is calculated using the length of the evaporative surface. For example, 70% on a length basis means that if the length of the evaporative surface is 10 mm, then 7 mm are surrounded by the evaporative assistance element.

A wick may comprise a porous or semi-porous substrate. The wick may be composed of various materials and methods of construction, including, but not limited to, bundled fibers which are compressed and/or formed into various shapes via overwrap (such as a non-woven sheet over-wrap) or made of sintered plastics such as PE, HDPE, or other polyolefins. The wick may be made from a plastic material such as polyethylene or a polyethylene blend.

### Evaporative Assistance Element

As previously discussed, the dispenser may include one or more evaporative assistance elements. The evaporative assistance element is used to assist with the evaporation of a volatile composition, preferably a fragrance, from the evaporative surface. For example, the evaporative assistance element may be selected from the group consisting of a heater, a fan, an agitation member or agitator that cause vibration, both powered agitator and manual agitator, or combinations thereof. The evaporative assistance element may also include a heating element to heat the liquid volatile composition, a chemical constituent to speed evaporation or evaporation rates, use of a chemically heated membrane to provide increased evaporation via exothermic reaction, or synergistic combinations thereof.

The evaporative assistance element can be coupled or mounted within the housing adjacent or in an air flow path to increase the air flow speed around the evaporative surface and out of the dispenser. The evaporative surface can be partially or fully surrounded or encircled by the evaporative assistance element. For example, as shown in FIGS. 5-7, the delivery engines 24, 26 extend into a channel 30a, 32a defined by the evaporative assistance elements 30, 32, which are shown as annular heaters. The relative positions of the evaporative assistance element(s) and the delivery engine(s) may be constant or variable. For example, the dispenser may be configured so that the evaporative assistance element or the delivery engine moves relative to the other so that there is a greater or smaller overlap between the evaporative assistance element and the delivery engine (e.g., more or less of the wick surface is exposed to a heater).

A volatile composition dispenser having an evaporative assistance element in the form of a heater may be configured to heat the evaporative surface to various temperatures. For example, the volatile composition dispenser may be configured such that the heater raises the temperature of the evaporative surface to a temperature of about 30° C. to about 150° C. The temperature gradient between the heater and the wick surface may be in a range of, for example, about 5° C. to about 20° C. or about 5° C. to about 10° C.

The heaters can comprise any suitable type of heater. Suitable evaporative assistance elements include a heater, UV/VIS lamp, resistor or array of resistors or any other heat source. An annular or hollow heater is preferred for use herein. The annular or hollow heater may extend entirely or partially around the evaporative surface of the delivery engine. For example, in some examples where the delivery engine extends through an annular or hollow heater, the heater may extend at least 180° to 360°, or from 2700 to 3600 around the evaporative surface. Especially preferred for use herein is a cylindrical evaporative surface surrounded by an annular heater. Additionally, one or more heaters may be used to extend around the evaporative surface. The heater is not, however, limited to an annular or hollow shape.

The proportion of the evaporative surface surrounded by the evaporative assistance element may be greater than in conventional dispensers, which increases the heating exposure area to enhance fragrance evaporation and reduce the air entry effectiveness to enable better chimney effect providing a greater fragrance emission. Surprisingly, this greater fragrance emission is achieved without changing the character of the fragrance.

As will be discussed in more detail below, the volatile composition dispenser may include a control system to control the evaporative assistance element.

### Switching Mechanism

With reference to FIG. 4, the volatile composition dispenser 10 may include a switching mechanism 40 that changes the volatile composition being emitted by the volatile composition dispenser 10. The switching mechanism 40 can comprise any suitable type of mechanism that causes the volatile composition dispenser to change the volatile composition being emitted. The switching mechanism may control the activation of the evaporative assistance elements, such as heaters, so that the heater will be turned on for the volatile composition that is desired to be emitted. Suitable switching mechanisms include, but are not limited to, analog timing circuitry, digital circuitry, combinations of analog and digital circuitry, microprocessors, and mechanical actuation switches such as shape memory alloys (NiTi wire) or bimetallic switches.

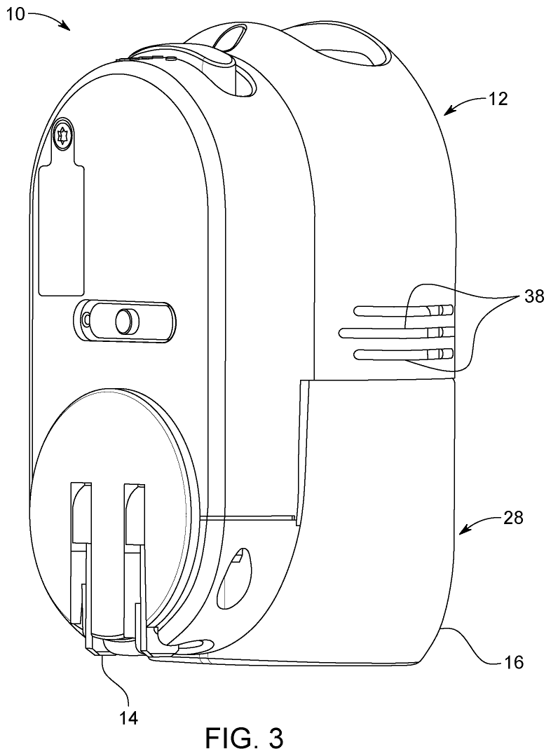

The switching mechanism 40 may comprise a combination analog and digital circuit in the form of a printed circuit board (or “PCB”). The circuit may include, for in a non-limiting example: a single-sided PC board; a capacitor; a pair of diodes; three transistors; five resistors; three counters; a third diode. Where the evaporative assistance elements are heaters, any suitable type of heater can be used, including but not limited to resistance heaters (several types of which are commercially available). The heaters, as well as the power source, shown as an electrical plug 14 in FIG. 3, may also be connected to the circuit board by wires. In an example, some or all of the components may be manually controllable in conjunction with a PCB or instead of a PCB.

In one example, a controller may control operation of the volatile composition dispenser by enabling one or more operational modes of the volatile composition dispenser based on data received by the controller from other components of the dispenser and/or from external components (e.g., input device, such as a smart phone).

The volatile composition dispenser may be capable of running programs that adjust to account for the particular volatile composition in use, which may be determined based on user input or a wireless communication tag. The reservoir or cartridges provided herein may include a wireless communication tag. The wireless communication tag may be a “smart” tag such as, for example, an NFC tag or an RFID tag, which can include both read and write functionality. The wireless communication tags can be sensed with a sensor (e.g., an antenna) and communicate with electrical components associated with the dispenser.

### Cartridge

Some parts of the volatile composition dispenser may be joined together to form a cartridge. For example, the reservoir(s), delivery engine(s), evaporative surface(s), and/or evaporative assistance element(s) may be joined together as one or more cartridges. With reference to FIG. 4, the reservoirs 16, 18, delivery engines 24, 26, including evaporative surfaces 24a, 26a, are connected together to form a cartridge 28. The volatile composition dispenser shown in FIG. 1 includes one cartridge 28, for example, but the volatile composition dispenser may be configured to contain more than one cartridge. A collar (e.g., collar 146) may be used to permanently or removably couple multiple reservoirs.

The cartridge(s) may be configured to be removably coupled to the housing. For example, at least a portion of the cartridge 28 may extend into an interior of the housing 12. The cartridge may entirely or partially be contained in the housing 12. For example, as shown in FIG. 1, a portion of the reservoirs 16, 18 or the cartridge 28 extend outside of the housing 12.

The cartridges or reservoirs may be replaceable in order to provide a reservoir with a new, different, or replacement volatile composition. Or, the reservoirs may be refillable and reused in the volatile composition dispenser in a new total emission program. The cartridge may be made of recyclable or sustainable materials.

### Air Flow

As described above, adjusting the configuration of the air inlet, air path through the dispenser, and air outlet can significantly enhance the consistency and volatile composition dispersion using the volatile composition dispenser. The addition of vents or openings on the sides, front and/or back of the housing to improve airflow within the dispenser can impact the room fill (both speed and consistency of experience). This increased dispersion surprisingly does not significantly impact the overall evaporation rate. For example, if the target rate was about 20 mg/Hr with no lower air inlet openings in the housing, it was observed that with the addition of inlet openings, the rate remains at about 20 mg/Hr while the room fill is improved dramatically.

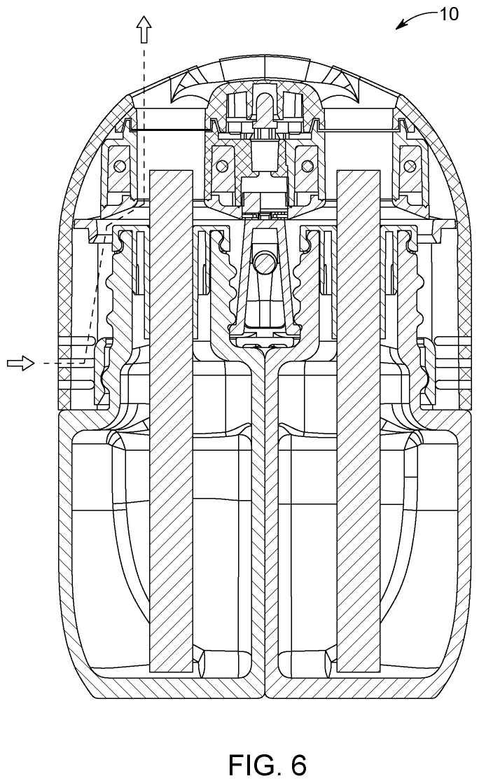

Referring to FIG. 6, an air flow path is shown through the dispenser 10. The air inlet opening, the channel created by the evaporative surface and the evaporative assistance element and the air outlet opening create an air flow path that improves the delivery of the volatile composition into the surrounding environment. In the present disclosure, the inlet opening is located upstream from the air entrance to the channel, and the entrance to the channel is located upstream from the air outlet opening. The dimensions and geometry of the inlet opening, the channel, and the outlet opening can play a role in air flow.

Preferably, the air inlet opening has a cross-sectional area (Aᵢₙₗₑₜ) of from about 250 mm² to about 1000 mm², preferably from about 300 mm² to about 800 mm², more preferably from about 400 mm² to about 600 mm². The air inlet opening can have one or more apertures. By “air inlet opening area” (Aᵢₙₗₑₜ) is herein meant the cross-section area of the inlet. The air inlet opening may be defined by one or more than one aperture such as, for example, one, two, three, four, five, six, seven, eight, nine, ten apertures, or more than two, more than four, or more than six apertures. In the case that the inlet has more than one qualifying aperture, the “air inlet opening area” is the sum of the cross-section area of each qualifying aperture. Where multiple apertures are included, the size and shape may be the same or may vary. For example, the cross-sectional area of each inlet aperture may be in a range of about 5 mm² to about 1,000 mm², about 5 mm² to about 500 mm², about 10 mm² to about 250 mm², about 25 mm² to about 60 mm², or about 40 mm² to about 50 mm². Only apertures that allow a flow path between the inlet and the channel, when the cartridge is inserted in the housing, are included in the measurement of the air inlet opening area. Gaps between the device housing and reservoir that allow air flow to the channel and that are greater than 100 mm² should be included in the inlet opening area measurement. For devices that have more than one channel, a separate inlet opening area can be measured for each channel. It is preferred to have more than one aperture.

The air outlet opening area (Aₒᵤₜₗₑₜ) may be in a range of, for example, about 30 mm² to about 100 mm², or about 40 mm² to about 80 mm², or about 40 mm² to about 50 mm², or about 75 mm² to about 85 mm².

The ratio of the air inlet opening area (Aᵢₙₗₑₜ) to the channel cross-section area (A_{channel}) may be in a range of, for example, about 5 to about 15, or about 9 to about 15. The ratio of air outlet opening area (Aₒᵤₜₗₑₜ) to the channel cross-section area (A_{channel}) may be in a range of, for example, about 1 to about 2.5, about 1 to about 2, or about 1.4 to about 1.75.

The position, size, and shape of the air inlet and air outlet may vary. The inlet or outlet apertures can have any geometry, such as circular, oval, racetrack or pill shaped, rectangular, etc. Preferred for use herein are inlet apertures having an elongated geometry, for example, with an aspect ratio of at least 1:2, preferably at least 1:5, or more preferably with an aspect ratio of about 1:10, or in a range of about 1:2 to about 1:20. The air inlet aperture(s) may be on a side, front, rear, or bottom of the housing. It is preferred that the air inlet apertures are positioned, relative to horizontal, below the channel of the evaporative assistance element(s). Preferred for use herein are outlet apertures having circular geometry.

For a volatile composition dispenser, the efficiency of the chimney effect inherent to an energized system is important to producing the desired performance—that is, delivery of the volatilized vapors into the space of effect (e.g., a room in a consumer home, office, or other such building). Defining the proper inlet airflow path and its resistance in such a system can improve the performance of such a dispenser—allowing the air to pass the region of applied energy (e.g., heater) and carry the volatile materials away from the outlet of the dispenser and into the targeted delivery space, which is external to the dispenser.

If an inlet opening area of a volatile composition dispenser is often very small and well below that of the outlet region, air flow into the device is impeded. When this occurs, one can still volatilize a perfume; however, the chimney effect is significantly limited and exhaust airflow is likewise reduced. By considering this need and ratio between the inlet and outlet portions of a device, it is desirable to create openings on the outside, lower portion of the housing such that the inlet opening area is greater than the channel cross section area, i.e., the annular region created by the evaporative assistance element (i.e., heater) surrounding the evaporative surface (i.e., the wick).

The air inlet or outlet may be unobstructed or partially obstructed. In configurations that include more than one air outlet or inlet, the size, shape, and degree of obstruction may vary between the outlets or inlets. For example, the inlet or outlet may include a baffle or a mesh component that affects the air flow exiting the dispenser. The effective size of the inlet or outlet may be variable based on, for example, a user setting. In some examples, the dispenser may be configured to selectively adjust the effective size of the air inlet(s) and outlet(s). In an example, may include one or more flaps or doors that can be moved relative to an air inlet or air outlet.

The interior walls defining the air flow path may be smooth or textured. For example, the interior walls may include dimples (e.g., like a golf ball) or internal grooves (e.g., rifling).

In FIGS. 5-7, the channel is illustrated as being substantially parallel to a longitudinal axis of the dispenser. In other configurations, the channel(s) may be positioned at an angle to the longitudinal axis of the dispenser.

The air flow path length and channel cross-sectional area, described below, are important dimensions for manipulating the chimney effect to provide the benefits of enhanced speed to room fill, and perfume consistency without increasing the power to the energy source nor increasing the rate of dispensing.

The channel cross-section area (A_{channel}) may be in a range of, for example, about 25 mm² to about 60 mm², about 25 mm² to about 35 mm², about 40 mm² to about 50 mm², or about 30 mm² to about 45 mm². The radial distance between the evaporative surface and the heater may be in a range of, for example, about 0.5 mm to about 2 mm, about 1.5 mm to about 2 mm, or about 1.7 mm to about 1.8 mm. The distance between the evaporative surface and the air outlet opening may be in a range of, for example, about 5 mm to about 25 mm, about 5 mm to about 20 mm, about 5 mm to about 10 mm, or about 15 mm to about 20 mm.

A temperature gradient between the air entering the air inlet opening(s) and the air exiting the air outlet opening(s) may be, for example, in a range of about 45° C. to about 65° C. A pressure drop between the air inlet and air outlet may be, for example, in a range of about 0.05 Pa to about 0.3 Pa or about 0.1 Pa to about 0.2 Pa.

### Measurements

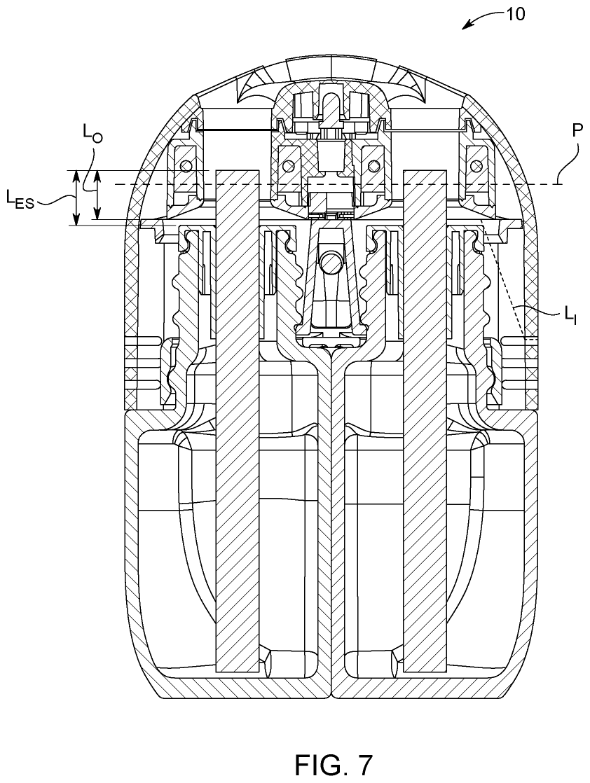

Referring to FIG. 7, various dimensions of the dispenser 10 are shown. Below are descriptions for methods of measuring relevant dimensions.

### Channel Cross-Section Area

The channel cross-section area is measured as the smallest cross-sectional area between the evaporative surface and overlapping evaporative assistance element. For example, with a device having a wick and an overlapping annular shaped heating element, the channel cross sectional area would be found by identifying the plane (P shown in FIG. 7) perpendicular to the wick where the area within the inner heater wall minus the area of the wick is the smallest area measurement. Further, the path that airflow traverses once inside the volatile composition dispenser will be such that the flow path follows into and through the cross sectional area which contains the energizing source (evaporative assistance element) as opposed to any other path through this region, airflow adjacent to this energizing source will not provide the benefits of enhanced distribution nor improved consistency within the treatment space outside of the dispenser. One might expect a significant impact on volatilization rate due to improved air velocity; however, it was surprisingly found that increasing the exit velocity and angle of departure did not significantly impact the volatilization/evaporation rate.

### Inlet Length

The inlet length, which may be considered a length of the air flow path, is defined as the shortest distance following the air flow path between an inlet opening and channel entrance. The entrance to the channel may be considered to be at a plane intersecting the bottom of the channel. Referring to FIG. 7, the inlet length (L_{I}) for the dispenser 10 is shown.

The inlet length (L_{I}) may be, for example, in a range of about 5 mm to about 20 mm, or about 10 mm to about 15 mm.

The ratio of Aᵢₙₗₑₜ to L_{I} may be in a range of, for example, about 15 mm to about 45 mm, about 20 mm to about 40 mm, or about 25 mm to about 40 mm.

For a dispensing device with an annular heater as the evaporative assistance element, the inlet length is measured according to the following instructions:
1. Start with an empty device manipulated in a way to make the below steps possible.
2. Obtain a thin string 0.5 meters long.
3. Keep one end of the string secured to the exterior of the device with tape.
4. Thread the other end of the string through the air inlet that is closest to either of the heaters (vent, bottom, hole etc.).
5. Thread the unsecured end of the floss through the heater, in the direction of airflow.
6. Place the refill into the device.
7. Now pull the string taut through device via the unsecured end.
8. Create a plane parallel to the one(s) utilized in determining the heater cross section area that contains the lowermost point of the heater.
9. Using a ruler as a straight-line guide and a drawing utensil, mark where the plane would intersect the wick.
10. Use the drawing device to mark the string where the string enters the interior of the device.
11. Take out refill.
12. Remove string from device.
13. Remove the taped side of the string from the device and lay the string on a flat surface.
14. Measure the distance between the two marks.
15. Record the measured distance in millimeters (mm).
16. Repeat steps 1-8 on various external entrances into the device and report the shortest distance achieved across all iterations.

### Evaporative Surface Length

To measure the length of the evaporative surface, the evaporative surface should be separated into two volumetrically equal parts starting at a point on the evaporative surface closest to the reservoir and ending at a point on the evaporative surface farthest from the reservoir. Referring to FIG. 7, the evaporative surface length (L_{ES}) is shown. The longest edge or curve of the separated evaporative surface should then be measured with a flexible ruler or measuring tape ensuring that the measuring device remains in contact with the evaporative surface following any curves or contours. The distance of the longest edge or curve measured should be recorded in millimeters (mm) as the length of the evaporative surface.

The evaporative surface length (L_{ES}) may be in a range of, for example, about 5 mm to about 20 mm, about 10 mm to about 20 mm, or about 12 mm to about 18 mm. The ratio of the inlet length (L_{I}) to evaporative surface length (L_{ES}) may be in a range of, for example, about 0.1 to about 1.7, about 0.5 to about 1.5, or about 0.7 to about 1.2.

### Channel Overlap Length

The channel overlap length is the length of the evaporative surface surrounded by the evaporative assistance element. Referring to FIG. 7, the channel overlap length (L_{O}) is shown as the length the channel extends over the exposed portion of the wick. The channel overlap length (L_{O}) may be in a range of, for example, about 5 mm to about 20 mm, preferably about 9 mm to 20 mm, more preferably about 10 mm to about 15 mm. The ratio of L_{O} to L_{I} may be in a range of, for example, about 0.6 to about 2.5, about 0.7 to about 2, or about 0.8 to about 1.5.

The channel overlap length of a dispensing device with a wick as the evaporative surface and an annular heater as the evaporative assistance element can be measured according to the following instructions:
1. Obtain a device that is manipulated in a way so that the steps below are possible.
2. Create a plane parallel to the one(s) utilized in determining the heater cross section area that contains the lowermost point of the heater.
3. Using a ruler as a straight-line guide and a drawing utensil, mark where the plane would intersect the wick.
4. Using an appropriate method, separate the wick into two volumetrically equal parts starting at a point on the wick closest to the reservoir and ending at a point on the wick farthest from the reservoir.
5. Using a flexible ruler or measuring tape, start where the plane intersects the wick and measure the longest edge/curve of the evaporative surface.
6. Ensure that the measuring device is in contact with the evaporative surface, following any curves or contours of the evaporative surface, to accurately capture the distance.
7. Record the distance in millimeters (mm).
8. The percent of the length of the wick surrounded by the heater can be calculated by dividing the channel overlap length (mm) by the length of the wick (mm).

The percent overlap (L_{O}/L_{ES}) of the channel overlap length by the length of the evaporative surface may be in a range of, for example, about 80% to about 95%, about 85% to about 95%, or about 90% to about 95%.

### Dispersion

Multiple factors affect the spread and noticeability of fragrance in an enclosed space. Such factors include, without limitation, the cone angle, maximum velocity, and average velocity of the air exiting the dispenser. These values may be measured using Background Oriented Schlieren (BOS) photography as discussed below.

The cone angle describes the angle of the scented air leaving the dispenser—it is the angle between two lines created perpendicular to the two most horizontal points of the plume. A cone angle may be too low such that it prevents adequate spread of fragrance and too high such that it could interfere with the dispenser. The cone angle may be in a range of, for example, about 250 to about 50°, about 30° to about 50°, about 350 to about 50°, or about 40° to about 50°. The ratio of the Aᵢₙₗₑₜ to A_{channel} ratio to the cone angle (units 1/°) may be in a range of, for example, about 0.15 to about 0.35, or about 0.2 to about 0.3. The ratio of the channel overlap length to the inlet length (L_{O}/L_{I}) ratio to the cone angle (units 1/°) may be in a range of, for example, about 0.015 to about 0.025, or about 0.18 to about 0.026. The ratio of percent overlap (L_{O}/L_{ES}) to the cone angle (units 1/°) may be, for example, in a range of about 0.01 to about 0.04, about 0.012 to about 0.024, or about 0.015 to about 0.022. The ratio of Aᵢₙₗₑₜ to L_{I} ratio to the cone angle (units mm/°) may be, for example, in a range of about 0.1 to about 1, about 0.5 to about 1, or about 0.6 to about 0.8. The ratio of Aᵢₙₗₑₜ to the cone angle (units mm²/°) may be, for example, in a range of about 7 to about 12, about 8.5 to about 10.5, or about 9 to about 10.

The average velocity is the average of the magnitudes of each velocity vector in the average velocity field (essentially the average of averages). Too high an average velocity may indicate the fragrance is reaching too far above the dispenser initially and not outwards into the room. The average velocity may be in a range of, for example, about 0.4 mm/s to about 1 mm/s, about 0.75 mm/s to about 1 mm/s, or about 0.8 mm/s to about 1 mm/s. The ratio of the Aᵢₙₗₑₜ to A_{channel} ratio to the average velocity (units s/mm) may be in a range of, for example, about 9 to about 35, about 10 to about 20, or about 10 to about 15. The ratio of the L_{O} to L_{I} ratio to the average velocity (units s/mm) may be in a range of, for example, about 0.1 to about 1.8, about 0.9 to about 1.21, or about 0.9 to 1.22. The ratio of Aₒᵤₜₗₑₜ to the average velocity (units mm*s) may be, for example, in a range of about 40 to about 250, or about 40 to about 100. The ratio of percent overlap (L_{O}/L_{ES}) to the average velocity (units s/mm) may be, for example, in a range of about 0.4 to about 3, about 0.8 to about 1.2, or about 0.9 to about 1.15. The ratio of Aᵢₙₗₑₜ to L_{I} ratio to the to the average velocity (units s) may be, for example, in a range of about 4 to about 50, about 30 to about 40, or about 34 to about 37.

The maximum velocity is the greatest velocity magnitude in the average velocity vector field. A maximum velocity that is too low could be indicative of a slow spread of fragrance, and one that is too high could indicate the scent would dissipate too quickly to be enjoyed. The maximum velocity may be in a range of, for example, about 2.5 mm/s to about 5 mm/s, about 3.5 mm/s to about 4.5 mm/s, or about 3.9 mm/s to about 4.4 mm/s.

When the air exiting the volatile composition dispenser is in laminar flow, it may be referred to as the plume. The plume height may be, for example, in a range of about 5 cm to about 10 cm above the air outlet.

### Flow Resistance

The air flow is mainly dependent on the heater temperature, air flow path length and cross section area. The heater temperature provides a driving force for the air flow, i.e., the buoyance-driven convection air flow. The length and cross-section area of the air flow path creates the resistance to the convection air flow. Minimizing the total flow resistance and maximizing the chimney effect around the wick may provide better perfume evaporation. Therefore, one design criterion is to minimize both inlet and outlet flow path resistance and to make the flow resistance occur in the heater and wick overlap region. The following equations are used to calculate the flow resistance in the inlet or the outlet region and their relative strength to the flow resistance in the heater and wick overlap region.

The flow resistance can be calculated using the formula:

$$Δ P = f {Q}^{2} \frac{L}{{A}^{2} D}$$

where ΔP (Pa) is the flow resistance at a constant flow rate, Q is the volumetric flow rate (m³/s), L is the flow path length (m), A is the flow cross section area (m²), D is the diameter of the flow channel (m), and f is the friction coefficient. In the laminar flow, the friction coefficient (f) is calculated using the following formula:

$$f = \frac{C}{Re} = \frac{C μ A}{ρ DQ}$$

where C is a constant, Re is the Reynolds number, μ is the absolute (dynamic) viscosity (Ns/m²), p is the density of the air (kg/m³).

The flow resistance can be calculated by the following substituted formula

$$Δ P = {c}^{′} Q \frac{L}{A {D}^{2}} \approx {c}^{″} Q \frac{L}{{A}^{2}}$$

where c′ and c″ are the constants.

The above equation can be used to calculate the ratio of the flow resistance through the air inlet flow path to the flow resistance through the channel as:

$$\frac{Δ {P}_{inlet}}{Δ {P}_{channel}} = \frac{{L}_{inlet}}{{L}_{channel}} {( \frac{{A}_{c hannel}}{{A}_{inlet}} )}^{2}$$

where:
   Lᵢₙₗₑₜ (L_{I}) is inlet length as measured according to instructions provided (m), L_{channel} (L_{O}) is the channel overlap length (in),
A_{channel} is channel cross-section area (m²), and
Aᵢₙₗₑₜ is the inlet opening area (m²).

The Reynolds number for the air flow through the channel may be, for example, in a range of about 13 to about 52, about 13 to about 35, or about 13 to about 20, or may be about 15. The air flow buoyancy flux (at the air outlet) may be, for example, in a range of about 4500 mm³/s to about 5500 mm³/s or may be about 5000 mm³/s. The air flow Rayleigh number in the heater and wick overlapping region may be, for example, in a range of about 500 to about 800.

The ratio of a flow resistance through the air inlet opening to a flow resistance through the channel may be, for example, less than 0.1, preferably less than 0.05, or more preferably less than 0.02 or may be in a range of, for example, about 0.001 to about 0.02, or about 0.001 to about 0.015.

### Volatile Composition

In certain examples, the liquid volatile composition can comprise a single chemical and/or a single material that is capable of entering the vapor phase or, more commonly, the liquid volatile composition can comprise a mixture of chemicals and/or materials that are capable of entering the vapor phase. In various examples, the liquid volatile composition can comprise substances that can function as air fresheners, deodorants, odor neutralizing materials, odor blocking materials, odor masking materials, aromatherapy materials, aromachology materials, essential oils, insecticides, pesticides, pheromones, medicinals, flavors and/or combinations thereof. In other various examples, the liquid volatile composition can comprise other materials that can act in their vapor phase to modify, enhance, and/or treat an atmosphere or an environment. The dispenser can be configured for use in any environment, such as a domestic environment, for example, and can be configured to dispense any suitable solutions, chemical, materials, and/or compositions.

Suitable liquid volatile compositions are described in U.S. Patent App. Pub. No. 2010/0308130 and U.S. Patent App. Pub. No. 2010/0308126, which are each incorporated herein by reference in their entireties. Especially suitable liquid volatile compositions are described in U.S. Pat. No. 10,322,198, which is incorporated herein by reference in its entirety, at column 3, line 45 to column 12, line 28.

The volatile liquid composition may comprise from greater than 10 wt %, alternatively greater than 20 wt %, alternatively greater than 30 wt %, alternatively greater than 40 wt %, alternatively greater than 50 wt %, alternatively greater than 60 wt %, alternatively greater than 70 wt %, alternatively greater than 85 wt %, of perfume raw materials, based on the total weight of the freshening volatile liquid composition.

The dispenser can comprise more than one reservoir, each hosting a different, slightly different, or the same volatile liquid composition. Each volatile composition can comprise a different, slightly different, or the same vapor pressure range, for example. This feature can be useful when a user wants to dispense a first dose amount of a first volatile composition and a second dose amount of a second volatile composition, for example. In an instance in which more than one volatile compound is within one reservoir, the volatile composition with the higher vapor pressure range may transform from a liquid phase into a vapor phase prior to the volatile composition with the lower vapor pressure range transforming into a vapor phase. In this circumstance, the volatile composition with the higher vapor pressure range would likely be dispensed first, while the volatile composition with the lower vapor pressure range would likely be dispensed second. In various examples, where different volatile compositions with different vapor pressure ranges are in separate reservoirs, the different volatile compositions can be dispensed from their respective reservoirs simultaneously, for example. As a result, various volatile compositions can be dispensed from the dispenser to create a mixture of scents, for example, if the volatile liquid composition is a fragrance.

### Use of the Dispenser

The dispenser can be configured to deliver a volatile liquid composition into the air with increased noticeability over the life of the volatile composition contained within a reservoir. It has been found that varying the energy applied to the volatile liquid composition over a total emission cycle can affect the consumer noticeability of the volatile composition over time. In particular, an initial energy boost period applied to the volatile composition, followed by a decrease in energy for an extended emission period, with successive energy boosts and variation in energy over a period results in improved noticeability of the volatile liquid composition by the user. Example emission programs are disclosed in U.S. Pat. No. 11,896,743. Variations on the emission programs disclosed in U.S. Pat. No. 11,896,743 include more frequent peaks and valleys. For example, the extended emission period may include one, two, three, or more than one energy boost to an energy lower than the energy boost immediately preceding the respective extended emission period. Where the extended emission period includes more than one peak, the peaks may vary. For example, the peaks may decrease in energy, have the same energy, or may increase in energy.

The evaporative assistance element may be configured to have various discrete emission periods, gaps in emission of any evaporative assistance elements, varying energy profiles over time, randomized energy profiles, simultaneous emission periods, and combinations thereof. Each of these methods of operation, either alone or in combination, may promote user noticeability of the volatile composition and/or reduce the likelihood of short-term or long-term habituation of the volatile composition.

The term “discrete emission period”, as used herein, refers to the individual time period that a given volatile composition is emitted in an emission sequence. This may correspond generally to the period of time that an evaporative assistance element is turned ON for a given fill of volatile composition, although there may be a slight lag between the operation of an evaporative assistance element and the emission of a volatile composition. The term “extended emission periods”, as used herein, includes a plurality of successive discrete emission periods that may be separated by gaps in operation where the evaporative assistance element is OFF.

The “total emission program” refers to the entire sequence, including all discrete emission periods and OFF times for gaps in emission that make up the energy boosts and extended emission periods, from beginning to end of life of a “filled” volume of volatile composition in a reservoir. “Fill” or “filled” as used herein refers to an amount of volatile composition that is intended to occupy the whole of or substantially the whole of the available volume in the reservoir, which excludes any volume occupied by any other elements of the volatile composition dispenser that may be disposed in the reservoir, such as the delivery engine. The reservoir will typically be occupied or filled to least 80%, 85%, 90%, or 95% volatile composition, of the total available volume of the reservoir. The total emission program is then designed to evaporate all or substantially all of the volatile composition in the reservoir.

The total emission program may be continuous. The term “continuous”, as used in reference to the emission program, means that there is a planned emission sequence over an entire period, once the program is initiated. This emission program can include periods, as noted above, where there are gaps in emission. This will still be considered to be a continuous emission program, although there will not necessarily be continuous emission of volatile compositions. It should be understood, however, that it is possible for the emission program to be interruptible by the user (e.g., turned off), if desired. Thus, the method can provide a user interface, and the user interface can provide a user with the ability to interrupt emission program. The emission program may be designed to run continuously, or substantially continuously until at least one of the volatile compositions is substantially depleted from the reservoir. It may be desirable for the emission program to run continuously until all of the volatile composition(s) are substantially depleted, and for this to occur at approximately the same time.

If the total emission program is disrupted, the dispenser may be configured with memory to record the last emission sequence that was initiated in the event that the volatile composition dispenser is disconnected from the power source. Once operation of the volatile composition dispenser is resumed, the memory of the last recorded sequence is recalled to return the total emission program to the correct emission sequence. The total emission program may only be restarted at the beginning of the program when a new or refilled reservoir/cartridge is installed into the housing.

The total emission program can be of any suitable length, including but not limited to 10 days, preferably about 15 days, preferably about 20 days, preferably about 25 days, preferably about 30 days, more preferably about 45 days, more preferably about 60 days, more preferably about 90 days, more preferably about 130 days, more preferably about 150 days, or shorter or longer periods, or any period between about 30 to about 150 days.

The discrete emission period for each evaporative assistance element in a volatile composition dispenser may be in the range of about 2 minutes to about 48 hours, about 5 minutes to about 48 hours, about 10 minutes to about 48 hours, about 15 minutes to about 48 hours, about 20 minutes to about 24 hours, about 30 minutes to about 8 hours, or about 45 minutes to about 4 hours. The higher the energy supplied by the evaporative assistance element, such as a higher temperature supplied by a heater, the shorter the discrete emission period that may be needed to provide a noticeable amount of volatile composition into the air.

During the discrete emission period for a particular evaporative assistance element, the evaporative assistance element will be continuously ON. In a volatile composition dispenser comprising more than one evaporative assistance element, the evaporative assistance element may have alternating discrete emission periods. In an alternating system, one evaporative assistance element may be turned ON while the other evaporative assistance element may be turned OFF. In an example with two evaporative assistance elements, each element may be alternatingly energized for a period of about 45 minutes. Or, one or more evaporative assistance element may be turned ON at a given time. The operation of two or more evaporative assistance element may overlap for a period of time. The greater the discrete emission period for each evaporative assistance element, the potential for higher concentrations of volatile composition in the surrounding space in order to increase user noticeability. There may also be time periods when all evaporative assistance elements are turned OFF. In an example with two evaporative assistance elements, each element may be alternatingly energized for a period of about 45 minutes with an intermediate period of about 5 minutes where no element is energized. Each evaporative assistance element may be configured to have the same discrete emission period, or some or all of the evaporative assistance elements may be configured to have different discrete emission periods.

Evaporation rates of the volatile composition from the evaporative member may be about 1 mg/hr to about 100 mg/hr, or about 3 mg/hr to about 60 mg/hr, or about 5 mg/hr to about 40 mg/hr, or about 10 mg/hr to about 40 mg/hr, or about 15 mg/hr to about 40 mg/hr.

Near the end of the total emission program, the volatile composition dispenser may operate at or near the maximum power output, such as maximum temperature or fan speed, until unplugged and a new cartridge or reservoir is installed.

The total emission program may be configured to turn OFF an evaporative assistance element when the volatile composition is depleted from the reservoir. For example, the evaporative assistance element may turn OFF after a predetermined time period for a given intensity setting. By turning OFF the evaporative assistance element, energy is not applied by the evaporative assistance element until the reservoir is refilled or replaced with a new fill of volatile composition.

As described above, the volatile composition dispenser may include a button to implement a temporary program. When a temporary program is activated (e.g., when a boost button is pressed), the current heating cycle is interrupted. The temporary program may operate for a defined period of time before the volatile composition dispenser returns to the previous heating cycle. The temporary program may have a discrete emission period(s) of about 1 minute to about 90 minutes, about 10 minutes to about 60 minutes, or about 30 minutes to about 50 minutes. For example, the temporary program may have a discrete emission period of about 30 minutes, about 40 minutes, or about 50 minutes.

In various examples, the controller can also be in electrical communication with a temperature sensor configured to sense the temperature of the atmosphere. In various examples, the temperature sensor can send a signal to the controller indicative of the temperature of the space, such that the controller can provide an output signal to a fan assembly or other various components of the dispenser, indicative of a volatile composition dosing amount for a particular temperature and/or temperature range. For example, higher temperature ranges may require greater dose amounts than lower temperature ranges to achieve the desired result. As a result, the dispenser can be power efficient such that it can maximize the life of the power source. The dispenser can be activated for 1-30 seconds, for example, and then be inactive for 10-200 seconds, for example. In other various examples, the dispenser can be set by a user to provide a desired intermittent dosing amount.

In various examples, the dispenser can comprise a sensor, such as a visible indicator, a light source, and/or an audible alert, configured to provide feedback to the user regarding the status of the dispenser or the room occupancy. In one example, the sensor can be used to alert the user of a property of the dispenser. In such examples, the feedback can be visual and/or audible and can indicate to the user, among other things, whether the dispenser is powered on, what volatile composition dosing amount is being dispensed, the power level of the power source, the amount, type, or level of the volatile composition within the delivery engine, and/or any other suitable feedback helpful or beneficial to the user. In various examples, the sensor can comprise one or more one indicators, such as a plurality of light sources, for example, electrically coupled to the controller and/or to the power source, and a translucent portion in the housing, such that the one or more indicators can be viewed by the user though the housing. In one example, the one or more indicators can be oriented in any suitable fashion such that various lights of the one or more indicators can emit visible light through the translucent portion of the housing, depending on what type of feedback is being provided to the user. In one example, the translucent portion of the housing can comprise any suitable shape and the one or more indicators can be arranged in a similar shape so that as one indicator, such as a light two or more light sources are powered or unpowered, the user is provided with at least a second feedback and so forth. In one example, at least one button is at least partially translucent allowing for one or more indicators to be viewable through the button.

With some liquid volatile compositions (for instance those comprising fragrances) it may be helpful to adjust a heater temperature, a fan speed, frequency of run time, or on/off time to compensate for the changing volatile composition formulation as high vapor pressure volatile composition raw materials will evaporate more quickly than low vapor pressure raw materials. In this case it may optionally be desirable to have the controller operate a fan more frequently as the volatile composition is evaporated over a period of many days. For instance, in one non-limiting example, the fan could run at 10% duty cycle for the first 10 days of usage but then slowly increase up to about 30% to about 40% duty cycle from days 11 up to 60 days. In this way, the fan frequency or duration can be increased to compensate for potentially a decline in fragrance intensity. By adjusting for the age, it is possible to deliver a more consistent scent intensity even as the fragrance amount and mixture of high to low vapor pressure components is changing with time. One non-limiting example of a means of keeping track of run time of the delivery engine is to monitor the voltage of the battery associated with the delivery engine. For instance, a fully charged Li-ion battery may be 3.60 Volts to about 3.7 Volts while a Li-ion battery that was used for thirty days might have a voltage of lower than 3.45 Volts. By monitoring the voltage of the battery, the controller can recognize the life of the delivery engine and can adjust operating conditions to deliver a consistent scent experience over the life of the delivery engine.

Another non-limiting example of a way to monitor time is to start a timer when the delivery engine is inserted and to keep track of hours/minutes that the evaporative assistance element has operated. As mentioned above, the run time could be adjusted as the product ages to deliver a more consistent scent experience.

In the instance where the battery voltage or run time is viewed as the indicator of the full life of the delivery engine, the controller could be programmed to provide a signal to the user such as turning on a red light or provide a flashing light to indicate that the reservoir is empty and/or needs to be replaced.

### Methods

Examples include a method of evaporating a liquid volatile composition into a space using an example dispenser disclosed herein. The method provides improved delivery of the volatile composition into a room.

### EXAMPLES

### Example 1

A prior art dispenser (Dispenser A) was compared with an example dispenser according to the invention (Dispenser B). Dispenser A was a Febreze® Plug “Fade Defy Plug” commercially available from The Procter & Gamble Co. in 2024. Dispenser B is the same design as Dispenser A modified with the addition of three horizontal vents on either side of the dispenser resulting in an overall cross-sectional area of 426 mm².

The dispensers were tested with 29 users. Two hotel rooms with identical layouts were utilized in this test, one with Dispenser A plugged in and the other with Dispenser B. Users were randomly assigned which room to enter first. The dispensers were turned on at the high setting about an hour before users entered the rooms. Users were asked to walk around the entirety of the room and then give an overall assessment. The user then proceeded to the other room and followed the same procedure.

The following metrics were used in the assessment: Which do you prefer (Overall preference), Which has the right level of scent intensity (Preference based on scent intensity), How consistently is the perfume distributed throughout the space (Room Fill/Travel) with results in Table 1 indicating the % of users that preferred A or B for each metric. An Overall Rating was determined by asking each user to assess each dispenser as poor (0), fair (25), good (50), very good (75), or excellent (100) and the dividing the total score by the total number of users. Intensity was determined by asking each user “How strong is the fragrance?” for each dispenser using a scale of 0-10 (0 representing none, 10 representing overpowering, with 6.5 to 7.5 being viewed as the “ideal intensity”) with results in Table 1 as the average score.

The results are shown in Table 1.

TABLE 1
Results

| | Dispenser A | Dispenser B |
|---|---|---|
| Overall Preference | 33 | 77 |
| Scent Intensity | 31 | 69 |
| Scent travel throughout the room | 38 | 62 |
| Overall Rating | 59 | 78** |
| Room Fill | 60 | 78** |
| Intensity | 5.6 | 6.6* |

**95% CL
*90% CL

The results in Table 1 show that the users significantly preferred Dispenser B and that Dispenser B exceeded the performance of Dispenser A on every key metric.

### Example 2

### Method to Evaluate Dispersion

A test was performed to evaluate the influence of the inlet area including the addition of apertures on the side of the housing. Background Oriented Schlieren (BOS) photography was used. This technique visualizes changes in the refractive index of air. These changes can be due to variations in temperature, pressure, or air composition. The Schlieren effect, similar to the heat haze observed over a sun-heated surface, is the fundamental principle behind BOS.

BOS works by focusing a camera on a distant background and observing distortions caused by refractive index changes. These distortions appear as shifts in the background pattern, providing a visual representation of the refractive index variations. Via this method, the entrained airflow exiting the device was observed and analysis software was used to assess the differences between the exiting air and the static background. The BOS algorithm calculates the displacement field from the captured images. This field represents the shifts in the background pattern caused by refractive index changes. From the displacement field, the strain field is derived. The strain field provides a quantitative measure of the deformation, indicating areas of high and low refractive index changes. The maximum normal strain gives displacement with reduced directional bias, enhancing the accuracy of the visualization.

The strain field is utilized to calculate the vector field for velocity for a given moment in time. For this experiment, 120 images were taken over 4 seconds using a LaVision Imager CX camera in combination with DaVis 11 image acquisition and processing software. The LaVision Imager CX camera was set up approximately 6 feet horizontally away from the device being analyzed at a same height (about 2.5 feet from the floor). The LaVision Imager CX camera and DaVis 11 software summarized data captured by each photo and created an average velocity vector field. The average velocity was calculated to be the average of the magnitudes of each velocity vector in the average velocity field (essentially the average of averages). The maximum velocity was the greatest velocity magnitude in the average velocity vector field. The cone angle was the angle between two lines created perpendicular to the two most horizontal points of the plume.

### Devices

The dispersion characteristics of Inventive Device 1 and Comparative Devices 1-6 were evaluated. Comparative Device 1 was a Febreze® Air Freshener Fade Defy PLUG sold by the Procter & Gamble Company. Inventive Device 1 had the same design as Comparative Device 1 except for the addition of vertical vents on the side of the device, which increased the inlet area and decreased the inlet length. Comparative Device 1 had an inlet area of 168 mm². The vertical vents on Inventive Device 1 (three on each side, six total) had a combined inlet area of 258 mm², leading to a total inlet area of 426 mm².

Comparative Device 2 was an Ambi Pur 3VOLUTION sold by The Procter & Gamble Company. Inventive Device 2 (see e.g., FIGS. 8 and 9), had the same design as Comparative Device 2 except for the addition of vertical vents on the side of the device, which increased the inlet area and decreased the inlet length. Comparative Device 2 had an inlet area of 386 mm², which includes three windows on the front side of the housing. The vertical vents on Inventive Device 2 (two on each side, four total) had a combined inlet area of 49 mm², leading to a total inlet area of 435 mm².

Inventive Device 1 and Comparative Device 1 have two reservoirs, two wicks, and two heaters with a distance between the wicks of 17.75 mm. Inventive Device 2 and Comparative Device 2 have three reservoirs, three wicks, and three heaters with a distance between the wicks of 13.5 mm.

Comparative Device 3 was an Ambi Pur Electric Room Diffuser Mono sold by The Procter & Gamble Company. While the heater settings (low, medium, high) on Comparative Device 1 and Comparative Device 2 are controlled by a button or slider, the heat settings for Comparative Device 3 are controlled by twisting a dial on the top of the device housing. In the configuration corresponding to the high setting, the heater is brought farther down the wick, leading to a greater channel overlap. In addition, the opening of the heater for Comparative Device 3 is closer to the inlet at this position, decreasing the inlet length. In the configuration corresponding to the low setting, the heater is raised up, leading to a smaller channel overlap and an increased inlet length.

Comparative Devices 4 and 5, respectively, were a Glade® PlugIns® Air Freshener Warmer and a Glade® PlugIns® Plus Air Freshener Warmer both sold by SC Johnson & Son, Inc. Comparative Devices 6 and 7, respectively, were an Air Wick® Plug-In Scented Oil Warmer and an Air Wick® Advanced Plug-In Scented Oil Warmer both sold by Reckitt. Tables 2A-2C describe various physical characteristics of Inventive Devices 1 and 2 and Comparative Devices 1-7.

TABLE 2A
Device Characteristics

| | | | | Radial | Air Outlet |
|---|---|---|---|---|---|
| | | Air Inlet | Channel | Distance | Opening |
| | Inlet | Opening | Cross- | between | Area (per air |
| | Length | Area | Section Area | Evap. | flow) |
| | (L_{I}) | (Aᵢₙₗₑₜ) | (A_{channel}) | Surface and | (Aₒᵤₜₗₑₜ) |
| | (mm) | (mm²) | (mm²) | Heater (mm) | (mm²) |
| Inventive Device 1 | 14.79 | 426 | 45 | 1.75 | 78.5 |
| Comparative Device 1 | 30.4 | 168 | 45 | 1.75 | 78.5 |
| Inventive Device 2 | 12 | 435 | 31.6 | 1.75 | 44.2 |
| Comparative Device 2 | 28 | 386 | 31.6 | 1.75 | 44.2 |
| Comparative Device 3 | 20.97 | 233 | 43 | 1.5 | 70.9 |
| (on “low”) | | | | | |
| Comparative Device 3 | 16.0 | 233 | 43 | 1.5 | 70.9 |
| (on “high”) | | | | | |
| Comparative Device 4 | 51.41 | 211 | 29 | 1.5 | 50.3 |
| Comparative Device 5 | 55.3 | 196 | 29 | 1.75 | 50.3 |
| Comparative Device 6 | 41.25 | 209 | 34 | 1.25 | 82.5 |
| Comparative Device 7 | 37.26 | 206 | 33 | 1.75 | 63.6 |

TABLE 2B
Device Characteristics

| | Distance | Evap. | Channel | | |
|---|---|---|---|---|---|
| | between Evap. | Surface* | Overlap | | Ratio |
| | Surface and | Length | Length | Percent | of Aᵢₙₗₑₜ |
| | Air Outlet | (L_{ES}) | (L_{O}) | Overlap | to L_{I} |
| | (mm) | (mm) | (mm) | (L_{O}/L_{ES}) | (mm) |
| Inventive Device 1 | 6.5 | 13.06 | 12 | 92% | 28.8 |
| Comparative Device 1 | 6.5 | 13.06 | 12 | 92% | 5.5 |
| Inventive Device 2 | 16.5 | 16.5 | 15 | 91% | 36.3 |
| Comparative Device 2 | 16.5 | 16.5 | 15 | 91% | 13.8 |
| Comparative Device 3 | 22 | 11.2 | 3.5 | 31% | 11.1 |
| (on “low”) | | | | | |
| Comparative Device 3 | 22 | 11.2 | 8.5 | 76% | 14.6 |
| (on “high”) | | | | | |
| Comparative Device 4 | 17.5 | 5.61 | 4.7 | 84% | 4.1 |
| Comparative Device 5 | 13.5 | 5.61 | 5 | 89% | 3.5 |
| Comparative Device 6 | 22.5 | 12.96 | 4 | 31% | 5.1 |
| Comparative Device 7 | 22.5 | 12.96 | 3.5 | 27% | 5.5 |

*All of the evaporative surfaces were wicks.

TABLE 2C
Device Characteristics

| | | | | | Ratio of flow resistance |
|---|---|---|---|---|---|
| | | | | | through the air inlet |
| | Ratio of | Ratio of | Ratio | | flow path to |
| | Aᵢₙₗₑₜ to | Aₒᵤₜₗₑₜ to | of L_{O} | Ratio of | flow resistance through |
| | A_{channel} | A_{channel} | to L_{I} | L_{I} to L_{ES} | channel |
| Inventive Device 1 | 9.46 | 1.74 | 0.81 | 1.13 | 0.014 |
| Comparative Device 1 | 3.73 | 1.74 | 0.39 | 2.33 | 0.18 |
| Inventive Device 2 | 13.8 | 1.40 | 1.25 | 0.73 | 0.004 |
| Comparative Device 2 | 12.2 | 1.40 | 0.54 | 1.70 | 0.01 |
| Comparative Device 3 | 5.42 | 1.65 | 0.17 | 1.87 | 0.2 |
| (on “low”) | | | | | |
| Comparative Device 3 | 5.42 | 1.65 | 0.53 | 1.43 | 0.07 |
| (on “high”) | | | | | |
| Comparative Device 4 | 7.26 | 1.73 | 0.09 | 9.16 | 0.21 |
| Comparative Device 5 | 6.76 | 1.73 | 0.09 | 9.86 | 0.24 |
| Comparative Device 6 | 6.14 | 2.43 | 0.10 | 3.18 | 0.27 |
| Comparative Device 7 | 6.24 | 1.93 | 0.09 | 2.88 | 0.29 |

### Dispersion Results

The dispersion of Inventive Devices 1 and 2 and Comparative Devices 1-7 was evaluated. Inventive Device 1 and Comparative Device 1 had the same temperature profile and power level and used the same perfume. The temperature of Inventive Device 1 and Comparative Device 1 ranged from about 51° C. to about 76° C. Similarly, Inventive Device 2 and Comparative Device 2 had the same temperature profile and power level and used the same perfume. The temperature of Inventive Device 2 and Comparative Device 2 ranged from about 70° C. to about 95° C. Comparative Device 3 ran at a temperature of about 80° C. Comparative Devices 4 and 5 had a low setting of about 61° C. and a high setting of about 75° C. Comparative Devices 6 and 7 had a temperature range of about 61° C. to about 84° C. on Day 1 at all settings.

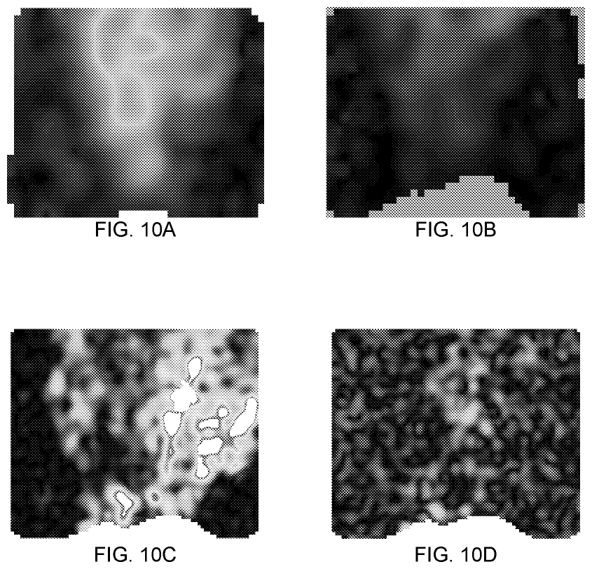

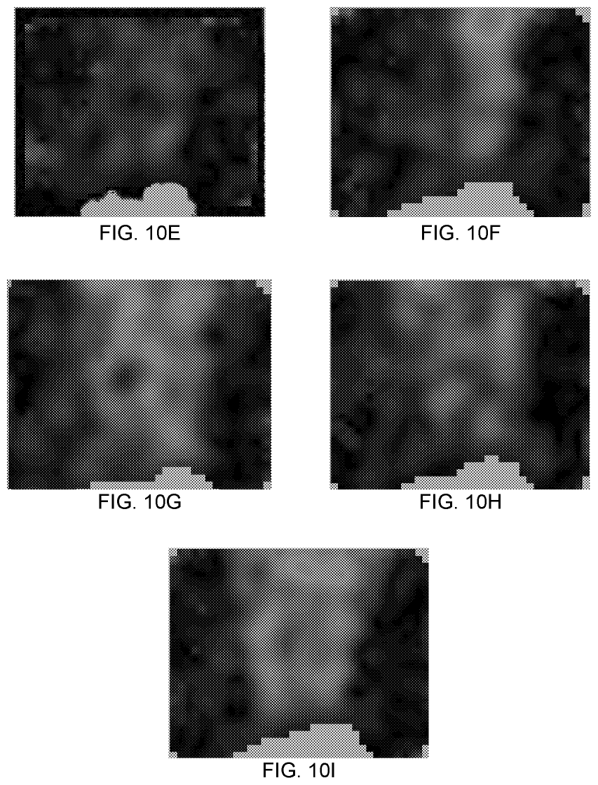

Table 3 shows the dispersion results including measurements of cone angle, maximum velocity (mm/s), and average velocity (mm/s). Additionally, FIGS. 10A-10I are Background Oriented Schlieren (BOS) photographs of the air exiting the dispenser outlet(s). In the photos, a whiter or brighter area indicates air that is moving at a faster velocity (i.e., the brighter, the faster).

TABLE 3
Dispersion Results for Evaluated Devices

| | | | Max | Average |
|---|---|---|---|---|
| | | Cone Angle | Velocity | Velocity |
| Device | Figure | (°) | (mm/s) | (mm/s) |
| Inventive Device 1 | 10A | 43 | 4.3 | 0.826 |
| Comparative Device 1 | 10B | 34 | 2.7 | 0.316 |
| Inventive Device 2 | 10C | 48 | 3.9 | 0.989 |
| Comparative Device 2 | 10D | 34 | 2.2 | 0.366 |
| Comparative Device 3 | 10E | 31 | 1.4 | 0.312 |
| (high setting) | | | | |
| Comparative Device 4 | 10F | 25 | 4 | 0.458 |
| Comparative Device 5 | 10G | 28 | 3.1 | 0.720 |
| Comparative Device 6 | 10H | 27 | 2.8 | 0.518 |
| Comparative Device 7 | 10I | 26 | 3.3 | 0.678 |

As described above, Inventive Devices 1 and 2 were the same as Comparative Devices 1 and 2 except for the addition of inlet vents. The results demonstrated that the addition of inlet vents had a surprising, significant positive impact on both the cone angle of release and the speed of the exiting airflow. Inventive Devices 1 and 2 each had an improved cone angle of 43° and 48°, respectively, compared to Comparative Devices 1 and 2 at 34° each. The larger the cone angle, the wider the spread of fragrance in a space.

Inventive Devices 1 and 2 each also had an improved maximum velocity of 4.3 mm/s and 3.9 mm/s, respectively, compared to Comparative Devices 1 and 2 at 2.7 mm/s and 2.2 mm/s, respectively. Similarly, Inventive Devices 1 and 2 each also had an improved average velocity relative to the average velocity of Comparative Devices 1 and 2. The plumes in FIGS. 10A and 10C (Inventive Devices 1 and 2) have a whiter area than those in FIGS. 10B and 10D-10I (Comparative Devices 1-7) confirming the improved velocity.

Tables 4A and 4B provide various device characteristics in relation to the dispersion results.

TABLE 4A
Device and Dispersion Characteristics

| | Ratio of | Ratio of | Ratio | | |
|---|---|---|---|---|---|
| | Ainlet | Aᵢₙₗₑₜ | of L_{O} | Ratio of | |
| | to A_{channel} | to A_{channel} | to L_{I} | L_{O} to L_{I} | Ratio of Air |
| | Ratio to | Ratio to | Ratio to | Ratio to | Outlet Area |
| | Cone | Average | Cone | Avg | to Average |
| | Angle | Velocity | Angle | Velocity | Velocity |
| Device | (1/°) | (s/mm) | (1/°) | (s/mm) | (mm*s) |
| Inventive Device 1 | 0.220 | 11.45 | 0.019 | 0.98 | 95 |
| Comparative Device 1 | 0.110 | 11.80 | 0.011 | 1.23 | 248 |
| Inventive Device 2 | 0.288 | 13.95 | 0.025 | 1.21 | 45 |
| Comparative Device 2 | 0.359 | 33.33 | 0.016 | 1.45 | 121 |
| Comparative Device 3 | 0.175 | 17.37 | 0.017 | 1.70 | 227 |
| (high setting) | | | | | |
| Comparative Device 4 | 0.290 | 15.85 | 0.004 | 0.20 | 110 |
| Comparative Device 5 | 0.241 | 9.39 | 0.003 | 0.13 | 70 |
| Comparative Device 6 | 0.227 | 11.85 | 0.004 | 0.19 | 159 |
| Comparative Device 7 | 0.240 | 9.20 | 0.003 | 0.13 | 94 |

TABLE 4B
Device and Dispersion Characteristics

| | Ratio of | Ratio of | | Ratio | |
|---|---|---|---|---|---|
| | Percent | Percent | Ratio | of Aᵢₙₗₑₜ | |
| | Overlap | Overlap | of Aᵢₙₗₑₜ | to L_{I} | Ratio of |
| | (L_{O}/L_{ES}) | (L_{O}/L_{ES}) | to L_{I} Ratio | Ratio to | Aᵢₙₗₑₜ to |
| | to Cone | to Avg | to Cone | Avg | Cone |
| | Angle | Velocity | Angle | Velocity | Angle |
| Device | (1/°) | (s/mm) | (mm/°) | (s) | (mm²/°) |
| Inventive Device 1 | 0.021 | 1.11 | 0.67 | 34.87 | 9.91 |
| Comparative Device 1 | 0.027 | 2.91 | 0.16 | 17.49 | 4.94 |
| Inventive Device 2 | 0.019 | 0.92 | 0.76 | 36.65 | 9.06 |
| Comparative Device 2 | 0.027 | 2.49 | 0.41 | 37.67 | 11.35 |
| Comparative Device 3 | 0.025 | 2.44 | 0.47 | 46.67 | 7.52 |
| (high setting) | | | | | |
| Comparative Device 4 | 0.034 | 1.83 | 0.16 | 8.96 | 8.44 |
| Comparative Device 5 | 0.032 | 1.24 | 0.13 | 4.92 | 7.00 |
| Comparative Device 6 | 0.011 | 0.60 | 0.19 | 9.78 | 7.74 |
| Comparative Device 7 | 0.010 | 0.40 | 0.21 | 8.15 | 7.92 |

### Example 3

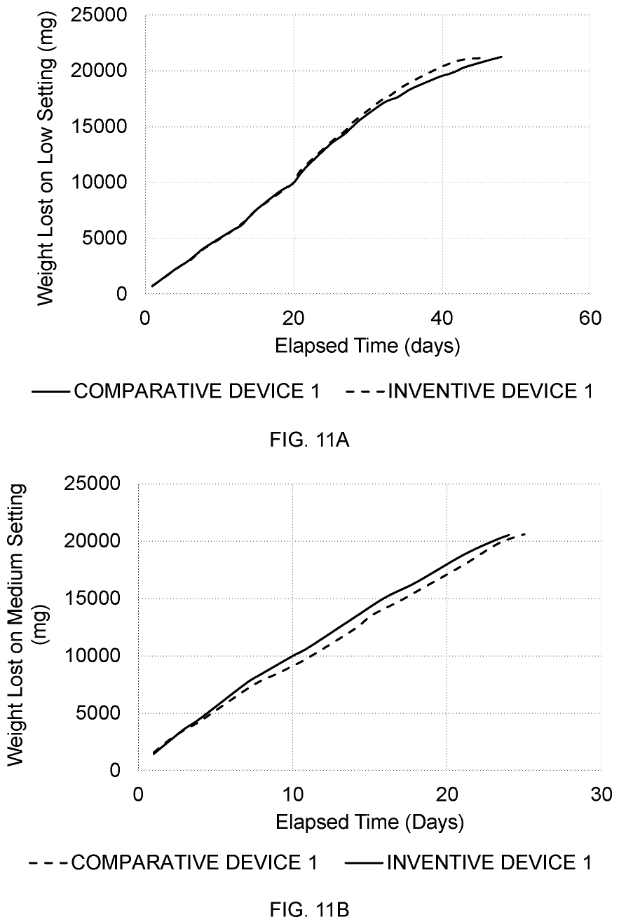

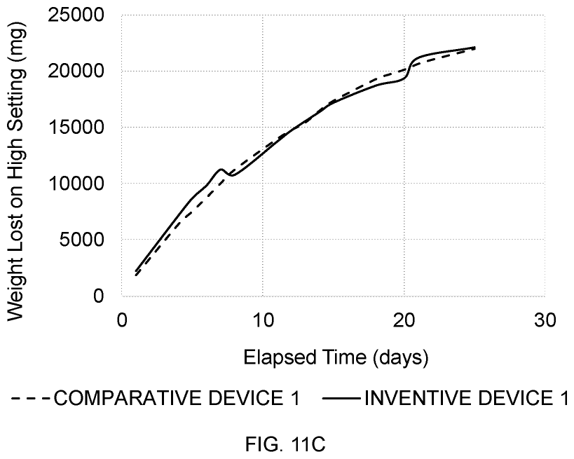

Inventive Device 1 and Comparative Device 1 were analyzed to determine whether the difference in dispersion for Inventive Device 1 and Comparative Device 1 resulted from the amount of volatile composition being dispensed. The volatile composition weight loss over time was analyzed for Comparative Device 1 and Inventive Device 1 at three different heat settings. Inventive Device 1 and Comparative Device 1 had the same temperature and power profiles for Low, Medium, and High settings and used the same perfume. The weight loss of the perfume over time on the Low, Medium, and High settings are shown in FIG. 11A-11C, respectively. As can be seen, there was not a significant difference in the weight loss profiles for the Inventive Device 1 and Comparative Device 1. In view of Example 2 where Inventive Device 1 showed improved dispersion, these weight loss results demonstrate that the dispensing effect of Inventive Device 1 was improved without significantly increasing the amount of volatile composition dispensed. In other words, the dispersion was not improved due to an increase in the volume of volatile composition being dispensed.

### COMBINATIONS

A. A dispenser for a volatile liquid composition, the dispenser comprising:
a housing comprising an air inlet opening and an air outlet opening, the housing being configured to contain a reservoir and a delivery engine;
an evaporative assistance element, wherein the evaporative assistance element defines a channel, and
wherein the dispenser defines an upward air flow path from the inlet opening through the channel and through the outlet opening,
wherein the channel has a cross-section area in a range of about 35 mm² to about 65 mm² and the air inlet opening has an area in a range of about 400 mm² to about 600 mm².

B. The dispenser as disclosed in A, further comprising the reservoir for containing the volatile liquid composition and the delivery engine in fluid communication with the volatile liquid composition, wherein the delivery engine comprises an evaporative surface at least partially extending through the channel.

C. The dispenser as disclosed in A or B, wherein a cartridge comprises the reservoir and the delivery engine, and the cartridge is removably coupled to the housing.

D. A dispenser for a volatile liquid composition, the dispenser comprising:
a housing comprising an air inlet opening and an air outlet opening;
a reservoir containing the volatile liquid composition;
an evaporative assistance element defining a channel; and
a delivery engine in fluid communication with the volatile liquid composition, the delivery engine comprises an evaporative surface at least partially extending into the channel,
wherein the dispenser defines an upward air flow path from the inlet opening through the channel and through the outlet opening, and
wherein a ratio of a length of the evaporative surface overlapped by the evaporative assistance element to a shortest distance between the air inlet opening to the channel entrance is greater than 0.6, preferably greater than 0.7.

E. A dispenser for a volatile liquid composition, the dispenser comprising:
a housing comprising an air inlet opening and an air outlet opening;
a reservoir containing the volatile liquid composition;
an evaporative assistance element, wherein the evaporative assistance element defines a channel; and
a delivery engine in fluid communication with the volatile liquid composition, the delivery engine comprises an evaporative surface at least partially extending into the channel,
wherein the dispenser defines an upward air flow path from the inlet opening through the channel and through the outlet opening, and
wherein a ratio of a flow resistance through the air inlet flow path to a flow resistance through the channel is less than 0.05, preferably less than 0.02.

F. A dispenser as disclosed in any one of A-E, wherein a ratio of the length of the evaporative surface surrounded by the evaporative assistance element to the shortest distance between the air inlet opening to the channel entrance is greater than 2.5, preferably greater than 5.

G. A dispenser as disclosed in any one of A-F, wherein a ratio of a flow resistance through the air inlet opening to a flow resistance through the channel is less than 0.1, preferably less than 0.05.

H. A dispenser as disclosed in any one of A-G, wherein a length of at least 9 mm of the evaporative surface is overlapped by the evaporative assistance element.

I. A dispenser as disclosed in any one of A-H, wherein a length of from about 9 mm to about 20 mm of the evaporative surface is overlapped by the evaporative assistance element.

J. A dispenser as disclosed in any one of A-I, wherein the inlet opening area is at least 250 mm², preferably at least 400 mm².

K. A dispenser as disclosed in any one of A-J, wherein the inlet opening area comprises one or more apertures.

L. A dispenser as disclosed in any one of A-K, wherein the inlet opening area comprises one or more apertures and the apertures are situated one above another.

M. A dispenser as disclosed in any one of A-L, wherein the inlet opening area comprises one or more apertures and the apertures have elongated geometry.

N. A dispenser as disclosed in any one of A-M, wherein the channel has a cross-section area of at least 40 mm².

O. A dispenser as disclosed in any one of A-N, wherein the evaporative surface and the evaporative assistance element have an annular arrangement.

P. A dispenser as disclosed in any one of A-O, wherein the evaporative surface is a wick, preferably having a diameter of from about 5 mm to about 10 mm.

Q. A dispenser as disclosed in any one of A-P, wherein the evaporative assistance element is a heater, preferably having an annular arrangement.

R. A dispenser as disclosed in any one of A-Q, wherein the dispenser is an energized system.

S. A dispenser as disclosed in any one of A-R, wherein the dispenser is a plug in system.

T. A dispenser for a volatile liquid composition, the dispenser comprising:
a housing comprising an air inlet opening and an air outlet opening wherein the housing retains:
   a) a reservoir holding the liquid composition;
   b) a delivery engine in fluid communication with the liquid composition, the delivery engine comprises an evaporative surface; and
   c) an evaporative assistance element,
   the evaporative assistance element partially surrounding the evaporative surface to define a channel
   wherein the inlet opening, the channel and the outlet opening define an upwards air flow path
   wherein a ratio of a length of the evaporative surface overlapped by the evaporative assistance element to a shortest distance between the air inlet opening to an entrance to the channel is greater than 0.6, preferably greater than 0.7.

U. The dispenser as disclosed in any one of A-T, wherein when the evaporative assistance element is energized, air exiting the air outlet opening has a cone angle in a range of about 350 to about 50°, preferably about 400 to about 50°.

V. The dispenser as disclosed in any one of A-U, wherein when the evaporative assistance element is energized, air exiting the air outlet opening has an average velocity in a range of about 0.4 mm/s to about 1 mm/s, preferably about 0.75 mm/s to about 1 mm/s, more preferably about 0.8 mm/s to about 1 mm/s.

W. The dispenser as disclosed in any one of A-V, wherein when the evaporative assistance element is energized, air exiting the air outlet opening has a maximum velocity in a range of about 2.5 mm/s to about 5 mm/s, preferably about 3.5 mm/s to about 4.5 mm/s, more preferably about 3.9 mm/s to about 4.4 mm/s.

X. The dispenser as disclosed in any one of A-W, wherein an inlet length (L_{I}) is in a range of about 5 mm to about 20 mm, preferably about 10 mm to about 15 mm.

Y. The dispenser as disclosed in any one of A-X, wherein an evaporative surface length (L_{ES}) is in a range of about 5 mm to about 20 mm, preferably about 10 mm to about 20 mm, more preferably about 12 mm to about 18 mm.

Z. The dispenser as disclosed in any one of A-Y, wherein a ratio of the inlet length (L_{I}) to the evaporative surface length (L_{ES}) is in a range of about 0.1 to about 1.7, preferably about 0.5 to about 1.5, more preferably about 0.7 to about 1.2.

AA. The dispenser as disclosed in any one of A-Z, wherein a ratio of an air inlet opening area (Aᵢₙₗₑₜ) to the inlet length (L_{I}) is in a range of about 15 mm to about 45 mm, preferably about 20 mm to about 40 mm, more preferably about 25 mm to about 40 mm.

BB. The dispenser as disclosed in any one of A-AA, wherein a percent overlap (L_{O}/L_{ES}) of the channel overlap length by the evaporative surface length is in a range of about 80% to about 95%, preferably about 85% to about 95%, more preferably about 90% to about 95%.

CC. The dispenser as disclosed in any one of A-BB, wherein a ratio of the Aᵢₙₗₑₜ to A_{channel} ratio to the cone angle (units 1/°) is in a range of about 0.15 to about 0.35, preferably about 0.2 to about 0.3.

DD. The dispenser as disclosed in any one of A-CC, wherein a ratio of the channel overlap length to the inlet length (L_{O}/L_{I}) ratio to the cone angle (units 1/°) is in a range of about 0.015 to about 0.025, preferably about 0.18 to about 0.026.

EE. The dispenser as disclosed in any one of A-DD, wherein a ratio of percent overlap (L_{O}/L_{ES}) to the cone angle (units 1/°) is in a range of about 0.01 to about 0.04, preferably about 0.012 to about 0.024, more preferably about 0.015 to about 0.022.

FF. The dispenser as disclosed in any one of A-EE, wherein a ratio of Aᵢₙₗₑₜ to L_{I} ratio to the cone angle (units mm/°) is in a range of about 0.1 to about 1, preferably about 0.5 to about 1, more preferably about 0.6 to about 0.8.

GG. The dispenser as disclosed in any one of A-FF, wherein a ratio of Aᵢₙₗₑₜ to the cone angle (units mm²/°) is in a range of about 7 to about 12, preferably about 8.5 to about 10.5, more preferably about 9 to about 10.

HH. The dispenser as disclosed in any one of A-GG, wherein a ratio of the Aᵢₙₗₑₜ to A_{channel} ratio to the average velocity (units s/mm) is in a range of about 9 to about 35, preferably about 10 to about 20, more preferably about 10 to about 15.

II. The dispenser as disclosed in any one of A-HH, wherein a ratio of the L_{O} to L_{I} ratio to the average velocity (units s/mm) is in a range of about 0.1 to about 1.8, preferably about 0.9 to about 1.21, more preferably about 0.9 to 1.22.

JJ. The dispenser as disclosed in any one of A-II, wherein a ratio of Aₒᵤₜₗₑₜ to the average velocity (units mm*s) is in a range of about 40 to about 250, preferably about 40 to about 100.

KK. The dispenser as disclosed in any one of A-JJ, wherein a ratio of percent overlap (L_{O}/L_{ES}) to the average velocity (units s/mm) is in a range of about 0.4 to about 3, preferably about 0.8 to about 1.2, more preferably about 0.9 to about 1.15.

LL. The dispenser as disclosed in any one of A-KK, wherein a ratio of Aᵢₙₗₑₜ to L_{I} ratio to the to the average velocity (units s) is in a range of about 4 to about 50, preferably about 30 to about 40, more preferably about 34 to about 37.

MM. The dispenser as disclosed in any one of A-LL, wherein when the evaporative assistance element is energized, air exiting the air outlet opening has:
a cone angle in a range of about 350 to about 50°, preferably about 40° to about 50°;
an average velocity in a range of about 0.4 mm/s to about 1 mm/s, preferably about 0.75 mm/s to about 1 mm/s, more preferably about 0.8 mm/s to about 1 mm/s; and/or
a maximum velocity in a range of about 2.5 mm/s to about 5 mm/s, preferably about 3.5 mm/s to about 4.5 mm/s, more preferably about 3.9 mm/s to about 4.4 mm/s.

NN. A method for delivering a volatile liquid composition, preferably a fragrance, using a dispenser as disclosed in any of A-MM.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”. Ratios disclosed herein may be shown in a format of “value” instead of “value:1” for the sake of simplicity.

As used in this specification and the appended claims, the articles “a”, “an”, and “the” each can include plural references unless the content clearly dictates otherwise. Thus, for example, “a component” may include one or more component unless specifically indicated.

All documents cited in the Detailed Description are, in relevant part, incorporated herein by reference; the citation of any document is not to be construed as an admission that it is prior art with respect to the present disclosure. To the extent that any meaning or definition of a term in this written document conflicts with any meaning or definition of the term in a document incorporated by reference, the meaning or definition assigned to the term in this written document shall govern.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

What is claimed is:

1. A dispenser for a volatile liquid composition, the dispenser comprising:
a housing comprising an air inlet opening and an air outlet opening, the housing being configured to contain a reservoir and a delivery engine; and
an evaporative assistance element, wherein the evaporative assistance element defines a channel,
wherein the dispenser defines an upward air flow path from the air inlet opening through the channel and through the air outlet opening, and
wherein the channel has a channel cross-section area in a range of about 35 mm² to about 65 mm² and the air inlet opening has an air inlet opening area in a range of about 400 mm² to about 600 mm².

2. The dispenser of claim 1, wherein a cartridge comprises the reservoir and the delivery engine, and the cartridge is removably coupled to the housing.

3. The dispenser of claim 1, further comprising the reservoir for containing the volatile liquid composition and the delivery engine in fluid communication with the volatile liquid composition, wherein the delivery engine comprises an evaporative surface at least partially extending through the channel.

4. The dispenser of claim 3, wherein a length of the evaporative surface overlapped by the evaporative assistance element (L_{O}) is in a range of about 5 mm to about 20 mm.

5. The dispenser of claim 3, wherein a ratio of a length of the evaporative surface overlapped by the evaporative assistance element (L_{O}) to a shortest distance between the air inlet opening to a channel entrance (L_{I}) is in a range of about 0.6 to about 2.5.

6. The dispenser of claim 3, wherein the evaporative surface and the evaporative assistance element have an annular arrangement.

7. The dispenser of claim 3, wherein the delivery engine is a wick having a diameter of from about 5 mm to about 10 mm.

8. The dispenser of claim 1, wherein the evaporative assistance element is a heater having an annular arrangement.

9. The dispenser of claim 8, wherein a radial distance between the evaporative surface and the heater is in a range of about 0.5 mm to about 2 mm.

10. The dispenser of claim 1, wherein a shortest distance between the air inlet opening to a channel entrance (LI) is in a range of about 5 mm to about 20 mm.

11. The dispenser of claim 1, wherein a ratio of a flow resistance through the air inlet opening to a flow resistance through the channel is in a range of about 0.001 to about 0.02.

12. The dispenser of claim 1, wherein the air inlet opening comprises more than one aperture having an elongated geometry.

13. The dispenser of claim 1, wherein the channel cross-section area is at least 40 mm².

14. The dispenser of claim 1, wherein a ratio of an air inlet opening area (Aᵢₙₗₑₜ) to the inlet length (L_{I}) is in a range of about 15 mm to about 45 mm.

15. The dispenser of claim 1, wherein a ratio of the inlet length (L_{I}) to the evaporative surface length (L_{ES}) is in a range of about 0.1 to about 1.7.

16. The dispenser of claim 1, wherein when the evaporative assistance element is energized, air exiting the air outlet opening has a cone angle in a range of about 350 to about 50°.

17. The dispenser of claim 1, wherein when the evaporative assistance element is energized, air exiting the air outlet opening has an average velocity in a range of about 0.4 mm/s to about 1 mm/s.

18. The dispenser of claim 1, wherein when the evaporative assistance element is energized, air exiting the air outlet opening has a maximum velocity in a range of about 2.5 mm/s to about 5 mm/s.

19. The dispenser of claim 1, further comprising an electrical plug.

20. A method for delivering a volatile liquid composition using the dispenser of claim 1.
